(19) United States
(12) Patent Application Publication
(10) Pub. No.: US 2011/0008304 A1
(43) Pub. Date: Jan. 13, 2011

(54) USE OF CELLS TO FACILITATE TARGETED DELIVERY OF NANOPARTICLE THERAPIES

(75) Inventors: Deryl Troyer, Manhattan, KS (US); Duy H. Hua, Manhattan, KS (US)

Correspondence Address: Casimir Jones, S.C., 2275 DEMING WAY, SUITE 310, MIDDLETON, WI, 53562, US

(73) Assignee: KANSAS STATE UNIVERSITY RESEARCH FOUNDATION, Manhattan, US (US)

(21) Appl. No.: 12/668,281

(22) PCT Filed: Jul. 9, 2008
(86) PCT No.: PCT/US08/69525

§ 371 Date: Sep. 27, 2010

Related U.S. Application Data
(60) Provisional application No. 60/958,753, filed on Jul. 9, 2007.

(51) Int. Cl. A61K 35/12; C12N 5/0789; A61K 31/522; A61K 47/08; A61K 38/00; A61P 37/00

(52) U.S. Cl. 424/93.71; 424/93.7; 435/325; 514/263.38; 514/772; 514/2.3

(57) ABSTRACT

The present invention is related to the use of cells, such as stem cells or immune system cells, to deliver nanogels comprising an active agent to a desired site in the body. The present invention utilizes cells as a delivery system for active agents that are difficult to deliver, such as active agents with poor solubility, that degrade easily, or that are toxic to the body. The nanogels are preferably non-toxic and can optionally include a lytic agent to program apoptosis of the cell to deliver the nanogel and active agent to a desired sire within the body.

This application claims the benefit of U.S. Provisional Application 60/958,753, filed Jul. 9, 2007, the contents of which are incorporated by reference in their entirety.

The present invention was made with government support under RO1 AG025500 awarded by the National Institutes of Health. The government has certain rights in the invention.

### FIELD OF THE INVENTION

The present invention is related to the use of cells, such as stem cells or immune system cells, to deliver nanogels comprising an active agent to a desired site in the body.

### BACKGROUND OF THE INVENTION

The ability to deliver therapeutic drugs, diagnostic agents, and macromolecules to the site of the disease remains a challenging problem. Uversky et al., J. Proteome. Res., 5, 2505 (2006); Torchilin, AAPS. J., 9, E128-E147 (2007). Nanotechnology is a rapidly emerging drug-delivery system that makes possible the controlled release of many small molecules. Cegnar et al., Expert Opinion on Biological Therapy., 5, 1557 (2005); Vinogradov, Curr. Pharm. Des, 12, 4703 (2006); Vinogradov et al., Bioconjug. Chem., 15, 50 (2004); Vinogradov et al., Adv. Drug Deliv. Rev., 54, 135 (2002). A number of approaches have been reported for the intra-cytoplasmic drug delivery including liposomes (Winterhalter and Lasic, Chem. Phys. Lipids, 64, 35 (1993); Torchilin, Nat. Rev. Drug Discov., 4, 145 (2005)), immunoliposomes, (Torchilin, Immunomethods., 4, 244 (1994)), micelles, (Wang et al., J. Drug Target, 13, 73 (2005)), lipoplexes/polyplexes, (Elouahabi and Ruysschaert, Mol. Ther., 11, 336 (2005)), and cell-penetrating peptides (Kerki et al., IUBMB. Life, 58, 7 (2006)).

PEI, a polyamine polymer, in combination with PEG, has been used as a nonviral gene delivery agent (Vinogrado et al., Bioconjug. Chem., 15, 50 (2004); Sung et al., Biol. Pharm. Bull., 26, 492 (2003); Boussif et al., PNAS, 92, 7297 (1995); Goula et al., Gene Ther, 5, 1291 (1998)). The polyamine polymer chains can be cross-linked to form a nanogel, which increases the stability of the complex (Vinogradov, Curr. Pharm. Des, 12, 4703 (2006); Vinogradov et al., Adv. Drug Deliv. Rev., 54, 135 (2002); Sung et al., Biol. Pharm. Bull., 26, 492 (2003); Vinogradov et al., J. Control Release, 107, 143 (2005); Vinogradov et al., J. Drug Target, 12, 517 (2004)]. PEI alone is very toxic to cells [Sung, et al., Biol. Pharm. Bull., 26, 492 (2003); Vinogradov et al., J. Control Release, 107, 143 (2005); Dong et al., Acta Biochim. Biophys. Sin.(Shanghai), 38, 780 (2006)]; in order to reduce the cytoxicity, PEI is coupled with PEG which makes the compound more water soluble and less toxic [Vinogradov et al., Adv. Drug Deliv. Rev., 54, 135 (2002); Sung, et al., Biol. Pharm. Bull., 26, 492 (2003); Vinogradov et al., J. Drug Target, 12, 517 (2004); Vinogradov et al., Bioconjug. Chem., 9, 805 (1998); Erbacher et al., J. Gene Med., 1, 210 (1999)].

What is needed in the art is a system for delivering nanogel compositions to a desired site within the body.

### SUMMARY OF THE INVENTION

The present invention is related to the use of cells, such as stem cells or immune system cells, to deliver nanogels comprising an active agent to a desired site in the body. In some embodiments, the present invention provides compositions comprising an in vitro culture of cells comprising a nanogel comprising an active agent and a lytic agent, wherein the lytic agent is provided in an amount sufficient to cause lysis of said stem cells at a predetermined time. The present invention is not limited to the use of any particular type of cells. The use of a variety of cell types is contemplated. In some embodiments, the cells are stem cells. The present invention is not limited to the use of any particular type of stem cells. The use of a variety of stem cells is contemplated. In some embodiments, the stem cells are selected from the group consisting of pluripotent stem cells and multipotent stem cells. In some embodiments, the stem cells are selected from the group consisting of embryonic stem cells and adult stem cells. In some embodiments, the stem cells are umbilical cord matrix stem cells. In some embodiments, the cells are immune system cells. The present invention is not limited to the use of any particular type of immune system cells. In some embodiments, the immune system cells are selected from the group consisting of leukocytes and lymphocytes. In some embodiments, the leukocytes are selected from the neutrophils, macrophages, dendritic cells, mast cells, eosinophils, basophils, monocytes and natural killer cells. In some embodiments, the lymphocytes are selected from the group consisting of helper T cells, killer T cells, and B cells.

The present is not limited to the use of any particular lytic agent. The use of a variety of lytic agents is contemplated. In some embodiments, the lytic agent is a detergent or surfactant. The present invention is not limited to the use of any particular type of detergent or surfactant. In some embodiments, the surfactant or detergent is nonionic, cationic, or anionic. In some preferred embodiments, the detergent is selected from the group consisting of Triton X-100 and Tween-20. In some embodiments, the cells comprise a suicide gene and said lytic agent is a pro-drug that is activated by the gene product of the suicide gene. The present invention is not limited to the use of any particular suicide gene or prodrug. In some embodiments, the suicide gene is thymidine kinase and said pro-drug is ganciclivor.

The present invention is not limited to the use of nanogel formed from any particular polymer. The use of a nanogels formed from a variety of polymers is contemplated. In some embodiments, the nanogel comprises a polymer selected from the group consisting of PEG, PEI, PGA and PLA and combinations thereof. In some preferred embodiments, the nanogel is a PEG/PEI nanogel. In some preferred embodiments, the nanogel is non-toxic. In some preferred embodiments, the PEG/PEI nanogel has a methylene proton ratio (CH2O:CH2N) of about 6.0:1 to about 8.0:1. In some preferred embodiments, the non-toxic nanogel is non-toxic as determined by an MTT assay, wherein cells loaded with the nanogel exhibit greater than 80% viability 48, 72 or 96 hours after loading with the nanogel as measured by the MTT assay. In some embodiments, the predetermined time for cell lysis is from about 36 to 96 hours. In some preferred embodiments, greater than 50%, 60%, 70%, 80%, or 90% of the cells undergo lysis within the predetermined time range, for example, from about 36 to 96 hours.

The present invention is not limited to the use of any particular active agent. The use of a variety of active agents is contemplated. In some embodiments, the active agent is selected from the group consisting of a therapeutic protein, a therapeutic compound, an antibiotic compound, and an antiviral compound. The present invention is not limited to the use of any particular therapeutic protein. In some embodiments, the therapeutic protein is an antimicrobial polypeptide. The present invention is not limited to the use of any particular therapeutic compound. In some embodiments, the therapeutic compound is a chemotherapeutic compound.

In some embodiments, the nanogel comprises a blocking agent. The present invention is not limited to the use of any particular blocking agent. In some embodiments, the blocking agent is present in a sufficient concentration to block amino groups on said PEI so that said PEI is non-toxic to cells. In some embodiments, the nanogel further comprises PEG cross-linked with said PEI and a blocking moiety. In some embodiments, the blocking agent is selected from the group consisting of an alkyl moiety, and alkenyl moiety, an aryl moiety, and acetyl moiety, and rhodamine. In some embodiments, the blocking agent is attached to said nanogel via an amino group on said nanogel. In some embodiments, the nanogel composition further comprises a labeling agent. The present invention is not limited to the use of any particular type of labeling agent. In some embodiments, the labeling agent is selected from the group consisting of a fluorescent compound, a fluorescent protein, and a nanometallic particle, for example nanogold particles.

In some embodiments, the present provides a nanogel composition comprising a therapeutic agent and a lytic agent, wherein said lytic agent is provided in an amount sufficient to cause cell lysis at a predetermined time following introduction into a cell. The nanogel composition can optionally comprise a blocking agent and/or labeling agent as described above.

In some embodiments, the present invention provides a composition comprising an in vitro culture of stem cells, said cells comprising a nanogel comprising an active agent. The cells can optionally comprise a lytic agent and/or labeling agent, etc. as described above.

In some embodiments, the present invention provides a composition comprising an in vitro culture of immune system cells, said cells comprising a nanogel comprising an active agent. The cells can optionally comprise a lytic agent and/or labeling agent, etc. as described above.

In some embodiments, the present invention provides a process for making a targeted therapeutic cell composition comprising: providing a culture of cells and a nanogel comprising a therapeutic agent and a lytic agent, wherein said lytic agent is provided in an amount sufficient to cause lysis of said cells at a predetermined time; loading said nanogel into said cells to provide nanogel-loaded cells. The nanogel composition can optionally comprise a blocking agent and/or labeling agent as described above.

In some embodiments, the present invention provides non-toxic nanogel compositions comprising particles comprising PEI having a size of from about 0.1 to about 200 nm, wherein said particles are non-toxic when introduced into a cell. In some preferred embodiments, the non-toxic nanogel is non-toxic as determined by an MTT assay, wherein cells loaded with the nanogel exhibit greater than 80% viability 48, 72 or 96 hours after loading with the nanogel as measured by the MTT assay. In some embodiments, the non-toxic nanogel comprises a blocking agent. The present invention is not limited to the use of any particular blocking agent. In some embodiments, the blocking agent is present in a sufficient concentration to block amino groups on said PEI so that said PEI is non-toxic to cells. In some embodiments, the blocking agent is PEG and said PEG is present in said composition so that said nanogel has a methylene proton ratio (CH₂O:CH₂N) of about 6.0:1 to about 8.0:1. In some embodiments, the nanogel further comprises PEG cross-linked with said PEI and a blocking moiety. In some embodiments, the blocking agent is selected from the group consisting of an alkyl moiety, and alkenyl moiety, an aryl moiety, and acetyl moiety, and rhodamine. In some embodiments, the blocking agent is attached to said nanogel via an amino group on said nanogel. In some embodiments, the nanogel composition is lyophilized. In some embodiments, the nanogel composition further comprises a labeling agent. The present invention is not limited to the use of any particular type of labeling agent. In some embodiments, the labeling agent is selected from the group consisting of a fluorescent compound, a fluorescent protein, and a nanometallic particle, for example nanogold particles.

In some embodiments, the present invention provides methods for treating a subject comprising: administering to a subject in need of treatment the cell composition or nanogel composition as described above.

### DESCRIPTION OF THE FIGURES

FIG. 1. Synthesis and putative structure of rhodamine-labled nanogel PEG-PEI.

FIG. 2. Structure of AQ10

FIG. 3. The effect of altered methylene proton ratio in PEG-PEI on Pan 02 cell viability. Pan 02 cells were seeded in a 96 well plate and after reaching ˜70% confluency, the media was replaced with fresh medium containing nanogel PEG-PEI with two different ratio's of methylene proton at different concentrations and incubated for 48 hrs. *Significantly different from untreated cells, ^{†}significantly different from nanogel PEG:PEI (CH₂O:CH₂N=4:1).

FIG. 4. Dose effect of AQ10 on Pan 02 cell viability: Pan 02 cells were seeded in a 96 well plate and after reaching ˜70% confluency, the media was replaced with fresh medium containing DMSO (0.125, 0.25, 0.5, 1% (v/v)) or AQ10 (μM) dissolved in DMSO as indicated in the figure. The DMSO did not show any adverse effect on the cell growth. Following incubation for 48 hrs an MTT assay was performed. Cell proliferation assay showed that AQ10 significantly decreased the Pan 02 cell viability compared to untreated and DMSO treated Pan 02 cells. *Significantly different from untreated cells, ^{†}significantly different from DMSO treated cells.

FIG. 5. Dose effect of nanogel PEG-PEI and 1% AQ10-nanogel PEG-PEI on Pan 02 cell viability. Pan 02 cells were seeded in a 96 well plate and after reaching ˜70% confluency, the media was replaced with fresh medium containing nanogel PEG-PEI or AQ10-nanogel PEG-PEI at different concentrations. Following incubation for 48 hrs cell proliferation assays were performed. MTT assay results were shown in (panel A) and the hemocytometer-trypan blue exclusion results were shown in (panel B). *Significantly different from untreated cells, ^{†}significantly different from nanogel PEG-PEI alone treated cells.

FIG. 6. Scheme for preparation of non-toxic pegylated PEG-PEI nanogels.

FIG. 7. Scheme for preparation of non-toxic acetylated PEG-PEI nanogels.

FIG. 8. Scheme for preparation of non-toxic acylated PEG-PEI nanogels.

FIG. 9. Decreased cell number following exposure of TK+UCMS cells to the pro-drug, Ganciclovir at a dose range of 0 μM to 1600 μM concentration

FIG. 10. Nanoparticle loading kinetics over a period ranging from 30 minutes to 36 hours. These data show that the threshold loading of nanoparticles into UCMS.

FIG. 11. Effect on apoptosis following loading UCMS cells with nanogel containing various amounts of detergent.

FIG. 12. Effect on apoptosis following loading Pan 02 cells with nanogel containing various amounts of detergent.

FIG. 13. Effect of control PLGA nanogel on RUCS cell viability as assayed by an MTT assay.

FIG. 14. Effect of PLGA nanogel loaded with Etoposide on RUCS cell viability as assayed by an MTT assay.

FIG. 15. Effect of PLGA nanogel loaded with Triton-X on RUCS cell viability as assayed by an MTT assay.

FIG. 16. Effect of PLGA nanogel loaded with Etoposide and Triton-X on RUCS cell viability as assayed by an MTT assay.

FIG. 17. Effect of control PLGA nanogel on Pan 02 cell viability as assayed by an MTT assay.

FIG. 18. Effect of PLGA nanogel loaded with Etoposide on Pan 02 cell viability as assayed by an MTT assay.

FIG. 19. Effect of PLGA nanogel loaded with Triton-X on Pan 02 cell viability as assayed by an MTT assay.

FIG. 20. Effect of PLGA nanogel loaded with Etoposide and Triton-X on Pan 02 cell viability as assayed by an MTT assay.

### DEFINITIONS

As used herein, the term “nanogel” means a composition of hydrophilic nanoscale particles that are formed from a cross-linked polymer network. The particle size can be from about 0.1 nm or 1 nm to about less than 10, 20, 40, 50, 50, 70, 80, 90, 100, 200 or 500 nm.

As used herein, the term “non-toxic nanogel” means a nanogel that is not toxic to a cell upon loading into the cell as measured by an MTT assay, wherein cells loaded with the nanogel exhibit greater than 80% viability as measured by the MTT assay 48 hours after loading. A “non-toxic PEG-PEI nanogel” is a nanogel comprised of cross-linked PEG and PEI polymers that is not toxic to a cell upon loading into the cell as measured by an MTT assay, wherein cells loaded with the non-toxic PEG-PEI nanogel exhibit greater than 80% viability as measured by the MTT assay at least 48, 72, or 96 hours after loading.

As used herein, the term “stem cell” means a cell that has the ability to differentiate into one or more lineages.

As used herein, the term “multipotent” means the ability of a cell to differentiate into cells of a closely related family of cells.

As used herein, the term “pluripotent” means the ability of a cell to differentiate into the three main germ layers: endoderm, ectoderm, and mesoderm.

As used herein, the term “embryonic stem cells” means stem cells derived from an embryo.

As used herein, the term “adult stem cell” mean stem cells derived from an adult source.

As used herein, the term “umbilical cord matrix stem cell” means stem cells or a population of stem cells comprising stem cells that are isolated from the umbilical cord matrix, which is known as Wharton's jelly.

As used herein, the term “mesodermal cell line” means a cell line displaying phenotypic characteristics associated with mesodermal cells.

As used herein, the term “endodermal cell line” means a cell line displaying phenotypic characteristics normally associated with endodermal cells.

As used herein, the term “neural cell line” means a cell line displaying characteristics normally associated with neural cell lines. Examples of such characteristics include, but are not limited to, expression of GFAP, neuron-specific enolase, Neu-N, neurofilament-N, or tau.

As used herein, the term “immune system cells” means cells that are part of the active or passive immune system, including lymphocytes and leukocytes, respectively.

As used herein, the term “lytic agent” means a compound or other agent that causes lysis of a cell. For example, a lytic agent can be a chemical compound such as a surfactant, a peptide such as antimicrobial peptide, protein, or a combination of suicide gene and a pro-drug that interact to cause cell lysis.

As used herein, the term “surfactant” means a substance that, when dissolved in water, lowers the surface tension of the water and increases the solubility of organic compounds.

As used herein, the term “suicide gene” means a gene that when activated, causes a cell carrying the gene to kill itself via apoptosis in the presence of a pro-drug.

As used herein, the term “pro-drug” means a compound that is acted upon by the product of a suicide gene to make a drug that triggers apoptosis.

As used herein, an “active agent” is a substance that has biological activity in the body. Examples of active agents include, but are not limited to, therapeutic compounds, therapeutic proteins, antibiotic compounds, antiviral compounds, antineoplastic compounds, chemotherapeutic agents, and the like.

As used herein, the term “therapeutic compound” means a non-protein molecule that provides a therapeutic benefit when administered to a subject.

As used herein, the term “therapeutic protein” means a protein molecule that provides a therapeutic benefit when administered to a subject.

As used herein, the term “antibiotic” means a compound that destroys or prevents the growth of a bacteria.

As used herein, the term “antiviral” means a compound that destroys or prevents the growth of a virus.

As used herein, the term “chemotherapeutic agent” means a compound that destroys or prevents the growth of a tumor or cancerous cell.

The term “gene” refers to a nucleic acid (e.g., DNA) sequence that comprises coding sequences necessary for the production of a polypeptide or precursor. The polypeptide can be encoded by a full length coding sequence or by any portion of the coding sequence so long as the desired activity or functional properties (e.g., enzymatic activity, ligand binding, signal transduction, etc.) of the full-length or fragment are retained. The term also encompasses the coding region of a structural gene and the including sequences located adjacent to the coding region on both the 5′ and 3′ ends for a distance of about 1 kb on either end such that the gene corresponds to the length of the full-length mRNA. The sequences that are located 5′ of the coding region and which are present on the mRNA are referred to as 5′ untranslated sequences. The sequences that are located 3′ or downstream of the coding region and that are present on the mRNA are referred to as 3′ untranslated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

Where amino acid sequence is recited herein to refer to an amino acid sequence of a naturally occurring protein molecule, amino acid sequence and like terms, such as polypeptide or protein are not meant to limit the amino acid sequence to the complete, native amino acid sequence associated with the recited protein molecule.

As used herein, the terms “an oligonucleotide having a nucleotide sequence encoding a gene” and “polynucleotide having a nucleotide sequence encoding a gene,” means a nucleic acid sequence comprising the coding region of a gene or, in other words, the nucleic acid sequence that encodes a gene product. The coding region may be present in either a cDNA, genomic DNA, or RNA form. When present in a DNA form, the oligonucleotide or polynucleotide may be single-stranded (i.e., the sense strand) or double-stranded. Suitable control elements such as enhancers/promoters, splice junctions, polyadenylation signals, etc. may be placed in close proximity to the coding region of the gene if needed to permit proper initiation of transcription and/or correct processing of the primary RNA transcript. Alternatively, the coding region utilized in the expression vectors of the present invention may contain endogenous enhancers/promoters, splice junctions, intervening sequences, polyadenylation signals, etc. or a combination of both endogenous and exogenous control elements.

As used herein, the term “regulatory element” refers to a genetic element that controls some aspect of the expression of nucleic acid sequences. For example, a promoter is a regulatory element that facilitates the initiation of transcription of an operably linked coding region. Other regulatory elements include splicing signals, polyadenylation signals, termination signals, etc.

As used herein, the term “recombinant DNA molecule” as used herein refers to a DNA molecule that is comprised of segments of DNA joined together by means of molecular biological techniques.

As used herein, the term “purified” or “to purify” refers to the removal of contaminants from a sample.

As used herein, the term “exogenous gene” means a gene that is not normally present in a host cell or organism or is artificially introduced into a host cell or organism.

As used herein, the term “negative selectable marker” refers to a gene that encodes a protein that allows for negative selection. An example of a negative selectable maker is the thymidine kinase gene, which allows for selection with gancyclovir.

As used herein, the term “vector” is used in reference to nucleic acid molecules that transfer DNA segment(s) from one cell to another. The term “vehicle” is sometimes used interchangeably with “vector.”

The term “expression vector” as used herein refers to a recombinant DNA molecule containing a desired coding sequence and appropriate nucleic acid sequences necessary for the expression of the operably linked coding sequence in a particular host organism. Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals.

The term “transfection” as used herein refers to the introduction of foreign DNA into eukaryotic cells. Transfection may be accomplished by a variety of means known to the art including calcium phosphate-DNA co-precipitation, DEAE-dextran-mediated transfection, polybrene-mediated transfection, electroporation, microinjection, liposome fusion, lipofection, protoplast fusion, retroviral infection, and biolistics.

The term “stable transfection” or “stably transfected” refers to the introduction and integration of foreign DNA into the genome of the transfected cell. The term “stable transfectant” refers to a cell that has stably integrated foreign DNA into the genomic DNA.

The term “transient transfection” or “transiently transfected” refers to the introduction of foreign DNA into a cell where the foreign DNA fails to integrate into the genome of the transfected cell. The foreign DNA persists in the nucleus of the transfected cell for several days. During this time the foreign DNA is subject to the regulatory controls that govern the expression of endogenous genes in the chromosomes. The term “transient transfectant” refers to cells that have taken up foreign DNA but have failed to integrate this DNA.

### DESCRIPTION OF THE INVENTION

The present invention is related to the use of cells, such as stem cells or immune system cells, to deliver nanogels comprising an active agent to a desired site in the body. The present invention utilizes cells as a delivery system for active agents that are difficult to deliver, such as active agents with poor solubility, that degrade easily, or that are toxic to the body. This use of cells, such as stem cells or immune system cells, represents a paradigm shift as the cells are used as delivery vehicles for a therapeutic agent as opposed to being used for therapeutic purposes in and of themselves. Indeed, in preferred embodiments, the cells used as carriers for nanogels are induced to undergo apoptosis and/or lysis after migration to a particular site within the body so that the active agent contained within the nanogel is delivered to a particular cell-type or tissue.

Polyethylene glycol-polyethylenimine (PEG-PEI) nanogels have been used to deliver nucleic acids and oligonucleotides into cells. Such nanogels have not, however, been used to deliver small molecule drug compounds or proteins. The present invention discloses PEG-PEI nanogels synthesized with methylene proton ratios (CH₂O:CH₂N) in PEG-PEI ranging from ˜6.8:1 to 4:1 and less, as shown by ¹H NMR spectra. Various nanogels were synthesized with varying ratios of CH₂O:CH₂N (methylene proton) in PEG-PEI as shown by ¹H NMR spectra and tested their cytotoxicity using a rodent pancreatic adenocarcinoma cell line (Pan 02). The nanogel PEG-PEI with methylene proton ratio of 4:1 was strongly cytotoxic to Pan 02 cells in vitro, while the nanogel with the methylene proton ratio of 6.8:1 was not toxic. A novel anti-cancer drug, 6-(hydroxymethyl)-1,4-anthracenedione (AQ) analogue (AQ10) was incorporated into nontoxic nanogel PEG-PEI and tested the effect of AQ10 loaded nanogel PEG-PEI (AQ10-nanogel PEG-PEI) and AQ10 dissolved in DMSO on Pan 02 cell growth. The size of this AQ10-nanogel PEG-PEI was characterized using atomic force microscopy (AFM). The studies showed that the AQ10-nanogel PEG-PEI is readily taken up by Pan 02 cells. Growth attenuation of Pan 02 cells treated with AQ10-nanogel PEG-PEI was three to four times that of cells treated with AQ10 dissolved in DMSO. These results show that PEG-PEI, usually used to deliver nucleic acids into cells, can also be used to deliver an insoluble small molecule anticancer drug, AQ10. The present invention further discloses that the nanogels comprising the active agent can be taken up by cells, including stem cells such as umbilical cord matrix stem cells. When the cells are administered to a subject, the cells migrate to specific areas in the subject. In this manner, cells that are loaded with the nanogel/active agent composition allow the targeted delivery of the composition to particular cells or tissues within the body of a subject. The invention is described in more detail below.

The present invention further provides novel, non-toxic nanogel compositions. In preferred embodiments, the non-toxic nanogel compositions comprise PEG-PEI nanoparticles. The PEG-PEI nanogel has many advantages. The PEG-PEI nanogel is versatile. The PEG-PEI nanoparticles can be loaded with both hydrophobic drugs and hydrophilic drugs, and can be used to deliver nucleic acid, e.g., DNA, RNA, siRNA, etc. Also, drugs do not need be incorporated during fabrication as is the case for PLGA, for example. Hence, the PEG-PEI nanogel can be lyophilized and stockpiled for long periods of time and drugs added as needed, rather than having to make a new batch every time a new drug is to be added.

### 1. Cells for Delivering Nanogels Comprising an Active Agent

In some embodiments, the present invention provides compositions comprising cells, such as a population or in vitro culture of cells, further comprising a nanogel that comprises an active agent, and optionally, a lytic agent. The nanogels, active agents, and lytic agents are described in more detail below. In preferred embodiments, the cells are cultured in the presence of the nanogel so that the cells take up the nanogel. In some embodiments, the cells are derived from the subject in an autologous transplant therapy. In other embodiments, the cells are from another donor and used in an allogenic transplant therapy.

The present invention is not limited to the use of any particular type of cells. Indeed, the use of a variety of cells is contemplated. In some embodiments, the cells are stem cells. Suitable stem cells include embryonic cells, adult stem cells, and umbilical cord matrix stem cells. In other embodiments, the cells are immune system cells. In preferred embodiments, the cells, when introduced into a subject, migrate or are otherwise delivered to a particular area within the body. In some preferred embodiments, the cells undergo lysis at a desired site within the body.

In some embodiments, the stem cells are umbilical cord matrix stem cells (UCMS cells). UCMS cells isolated from Wharton's Jelly of the umbilical cord matrix. Methods for obtaining populations of UCMS cells are described in Mitchell et al., Stem Cells 21(1):50-60 (2003); Weiss et al., Stem Cells 24(3):781-92 (2005); and Troyer et al., Stem Cells 26(3):591-99 (2008). The umbilical cord contains an inexhaustible, non-controversial source of stem cells for therapy. Stem cells derived from human umbilical cord Wharton's Jelly, called umbilical cord matrix stem (UCMS) cells, are characterized. UCMS cells: 1) are isolated in large number; 2) are negative for CD34 and CD45, 3) grow robustly and can be frozen/thawed, 4) can be clonally expanded, and 5) can easily be engineered to express exogenous proteins. UCMS cells have genetic and surface markers of mesenchymal stem cells (positive for CD10, CD13, CD29, CD44, CD90, and negative for CD14, CD33, CD56, CD31, CD34, CD45 and HLA-DR), and appear to be stable in terms of their surface marker expression in early passage (passages 4-8). Unlike traditional mesenchymal stem cells derived from adult bone marrow stromal cells, small populations of UCMS cells express endoglin (SH2, CD105) and CD49e at passage 8. UCMS cells express growth factors and angiogenic factors suggesting that they may be used to treat neurodegenerative disease.

In some embodiments, the stem cells are pluripotent stem cells. Methods for obtaining pluripotent cells from a number of species, including monkeys, mice, rats, pigs, cattle and sheep have been previously described. See, e.g., U.S. Pat. Nos. 5,453,357; 5,523,226; 5,589,376; 5,340,740; and 5,166,065 (all of which are specifically incorporated herein by reference); as well as, Evans, et al., Theriogenology 33(1):125-128, 1990; Evans, et al., Theriogenology 33(1):125-128, 1990; Notarianni, et al., J. Reprod. Fertil. 41(Suppl.):51-56, 1990; Giles, et al., Mol. Reprod. Dev. 36:130-138, 1993; Graves, et al., Mol. Reprod. Dev. 36:424-433, 1993; Sukoyan, et al., Mol. Reprod. Dev. 33:418-431, 1992; Sukoyan, et al., Mol. Reprod. Dev. 36:148-158, 1993; Iannaccone, et al., Dev. Biol. 163:288-292, 1994; Evans & Kaufman, Nature 292:154-156, 1981; Martin, Proc Natl Acad Sci USA 78:7634-7638, 1981; Doetschmanet al. Dev Biol 127:224-227, 1988); Gileset al. Mol Reprod Dev 36:130-138, 1993; Graves & Moreadith, Mol Reprod Dev 36:424-433, 1993 and Bradley, et al., Nature 309:255-256, 1984.

Primate embryonic stem cells suitable for use in vivo are preferred. Primate embryonic stem cells may be obtained by the methods disclosed in U.S. Pat. Nos. 5,843,780 and 6,200,806, each of which is incorporated herein by reference. Primate (including human) stem cells may also be obtained from commercial sources such as WiCell, Madison, Wis. A preferable medium for isolation of embryonic stem cells is “ES medium.” ES medium consists of 80% Dulbecco's modified Eagle's medium (DMEM; no pyruvate, high glucose formulation, Gibco BRL), with 20% fetal bovine serum (FBS; Hyclone), 0.1 mM β-mercaptoethanol (Sigma), 1% non-essential amino acid stock (Gibco BRL). Preferably, fetal bovine serum batches are compared by testing clonal plating efficiency of a low passage mouse ES cell line (ESⱼₜ₃), a cell line developed just for the purpose of this test. FBS batches must be compared because it has been found that batches vary dramatically in their ability to support embryonic cell growth, but any other method of assaying the competence of FBS batches for support of embryonic cells will work as an alternative.

Primate ES cells are isolated on a confluent layer of murine embryonic fibroblast in the presence of ES cell medium. Embryonic fibroblasts are preferably obtained from 12 day old fetuses from outbred CF1 mice (SASCO), but other strains may be used as an alternative. Tissue culture dishes are preferably treated with 0.1% gelatin (type I; Sigma). Recovery of rhesus monkey embryos has been demonstrated, with recovery of an average 0.4 to 0.6 viable embryos per rhesus monkey per month, Seshagiri et al. Am J Primatol 29:81-91, 1993. Embryo collection from marmoset monkey is also well documented (Thomson et al. “Non-surgical uterine stage preimplantation embryo collection from the common marmoset,” J Med Primatol, 23:333-336 (1994)). Here, the zona pellucida is removed from blastocysts by brief exposure to pronase (Sigma). For immunosurgery, blastocysts are exposed to a 1:50 dilution of rabbit anti-marmoset spleen cell antiserum (for marmoset blastocysts) or a 1:50 dilution of rabbit anti-rhesus monkey (for rhesus monkey blastocysts) in DMEM for 30 minutes, then washed for 5 minutes three times in DMEM, then exposed to a 1:5 dilution of Guinea pig complement (Gibco) for 3 minutes.

After two further washes in DMEM, lysed trophectoderm cells are removed from the intact inner cell mass (ICM) by gentle pipetting, and the ICM plated on mouse inactivated (3000 rads gamma irradiation) embryonic fibroblasts. After 7-21 days, ICM-derived masses are removed from endoderm outgrowths with a micropipette with direct observation under a stereo microscope, exposed to 0.05% Trypsin-EDTA (Gibco) supplemented with 1% chicken serum for 3-5 minutes and gently dissociated by gentle pipetting through a flame polished micropipette. Dissociated cells are replated on embryonic feeder layers in fresh ES medium, and observed for colony formation. Colonies demonstrating ES-like morphology are individually selected, and split again as described above. The ES-like morphology is defined as compact colonies having a high nucleus to cytoplasm ratio and prominent nucleoli. Resulting ES cells are then routinely split by brief trypsinization or exposure to Dulbecco's Phosphate Buffered Saline (without calcium or magnesium and with 2 mM EDTA) every 1-2 weeks as the cultures become dense. Early passage cells are also frozen and stored in liquid nitrogen.

In some embodiments, the present invention provides compositions comprising adult stem cells. The adult stem cell is an undifferentiated (unspecialized) cell that is found in a differentiated (specialized) tissue; it can renew itself and become specialized to yield specialized cell types of the tissue from which it originated. These precursor cells exist within the differentiated tissues of the adult of all multicellular organisms in the animal kingdom as a community of cells dispersed throughout the tissue. Precursor cells derived from adults can be divided into three categories based on their potential for differentiation. These three categories of precursor cells are epiblast-like stem cells, germ layer lineage stem cells, and progenitor cells. Precursor cells have been isolated from a wide variety of tissues, including, but not limited to, skeletal muscle, dermis, fat, cardiac muscle, granulation tissue, periosteum, perichondrium, brain, meninges, nerve sheaths, ligaments, tendons, blood vessels, bone marrow, trachea, lungs, esophagus, stomach, liver, intestines, spleen, pancreas, kidney, urinary bladder, and testis. Precursor cells can be released from the connective tissue compartments throughout the body by mechanical disruption and/or enzymatic digestion and have been isolated from, but not limited to, newborns, adolescent, and geriatric mice, rats and humans, and adult rabbits, dogs, goats, sheep, and pigs.

The first category of precursor cells, epiblast-like stem cells (ELSCs), consists of a stem cell that will form cells from all three embryonic germ layer lineages. Stem cells from adult rats and stem cells from adult humans can be released from the connective tissue compartments throughout the body by mechanical disruption and/or enzymatic digestion. The stem cells from either adult rats or adult humans can be preferentially slow frozen and stored at −80° C.±5° C. using 7.5% ultra-pure dimethyl sulfoxide. Fast thawing of stem cells from both species from the frozen state to ambient temperature yields recovery rates exceeding 98%. These cells in the undifferentiated state express the Oct-3/4 gene that is characteristic of embryonic stem cells. ELSCs do not spontaneously differentiate in a serum free environment lacking progression agents, proliferation agents, lineage-induction agents, and/or inhibitory factors, such as recombinant human leukemia inhibitory factor (LIF), recombinant murine leukemia inhibitory factor (ESGRO), or recombinant human anti-differentiation factor (ADF). Embryonic stem cells spontaneously differentiate under these conditions. In contrast, ELSCs derived from both species remain quiescent unless acted upon by specific proliferative and/or inductive agents and/or environment.

ELSCs proliferate to form multiple confluent layers of cells in vitro in the presence of proliferation agents such as platelet-derived growth factors and respond to lineage-induction agents. ELSCs respond to hepatocyte growth factor by forming cells belonging to the endodermal lineage. Cell lines have expressed phenotypic markers for many discrete cell types of ectodermal, mesodermal, and endodermal origin when exposed to general and specific induction agents.

The second category of precursor cells consists of three separate stem cells. Each of the cells forms cells of a specific embryonic germ layer lineage (ectodermal stem cells, mesodermal stem cells and endodermal stem cells). When exposed to general and specific inductive agents, germ layer lineage ectodermal stem cells can differentiated into, for example, neuronal progenitor cells, neurons, ganglia, oligodendrocytes, astrocytes, synaptic vesicles, radial glial cells, and keratinocytes.

The third category of precursor cells present in adult tissues is composed of a multitude of multipotent, tripotent, bipotent, and unipotent progenitor cells. In solid tissues these cells are located near their respective differentiated cell types. Progenitor cells do not typically display phenotypic expression markers for pluripotent ELSCs, such as stage specific embryonic antigen-4, stage-specific embryonic antigen-1 or stage-specific embryonic antigen-3, or carcinoembryonic antigen cell adhesion molecule-1. Similarly, progenitor cells do not typically display phenotypic expression markers for germ layer lineage stem cells, such as nestin for cells of the ectodermal lineage or fetoprotein for cells of the endodermal lineage.

A progenitor cell may be multipotent, having the ability to form multiple cell types. A precursor cell of ectodermal origin residing in the adenohypophysisand designated the adenohypophyseal progenitor cell is an example of a multipotent progenitor cell. This cell will form gonadotrophs, somatotrophs, thyrotrophs, corticotrophs, and mammotrophs. Progenitor cells for particular cell lineages have unique profiles of cell surface cluster of differentiation (CD) markers and unique profiles of phenotypic differentiation expression markers. Progenitor cells do not typically spontaneously differentiate in serum-free defined medium in the absence of a differentiation agent, such as LIF or ADF. Thus, unlike embryonic stem cells which spontaneously differentiate under these conditions, progenitor cells remain quiescent unless acted upon by proliferative agents (such as platelet-derived growth factor) and/or progressive agents (such as insulin, insulin-like growth factor-I or insulin-like growth factor-II).

Progenitor cells can regulate their behavior according to changing demands such that after transplantation they activate from quiescence to proliferate and generate both new satellite cells and substantial amounts of new differentiated cells. For example, the contractile units of muscle are myofibers, elongated syncytial cells each containing many hundreds of postmitotic myonuclei. Satellite cells are resident beneath the basal lamina of myofibers and function as myogenic precursors during muscle regeneration. In response to muscle injury, satellite cells are activated, proliferate, and differentiate, during which they fuse together to repair or replace damaged myofibers. When satellite cells are removed from their myofibers by a non-enzymatic physical titration method, they retain their ability to generate substantial quantities of new muscle after grafting that they are not able to attain by enzymatic digestion. Conventional enzymatic disaggregation techniques impair myogenic potential. Collins and Partridge “Self-Renewal of the Adult Skeletal Muscle Satellite Cell” Cell Cycle 4:10, 1338-1341 (2005).

Accordingly, the present invention also contemplates the use of non-embryonic stem cells, such as those described above. In some embodiments, mesenchymal stem cells (MSCs) can be derived from marrow, periosteum, dermis and other tissues of mesodermal origin (See, e.g., U.S. Pat. Nos. 5,591,625 and 5,486,359, each of which is incorporated herein by reference). MSCs are the formative pluripotential blast cells that differentiate into the specific types of connective tissues (i.e. the tissues of the body that support the specialized elements; particularly adipose, areolar, osseous, cartilaginous, elastic, marrow stroma, muscle, and fibrous connective tissues) depending upon various in vivo or in vitro environmental influences. Although these cells are normally present at very low frequencies in bone marrow, various methods have been described for isolating, purifying, and greatly replicating the marrow-derived mesenchymal stems cells in culture, i.e. in vitro (See also U.S. Pat. Nos. 5,197,985 and 5,226,914 and PCT Publication No. WO 92/22584, each of which are incorporated herein by reference).

Various methods have also been described for the isolation of hematopoietic stem cells (See, e.g., U.S. Pat. Nos. 5,061,620; 5,750,397; 5,716,827 all of which are incorporated herein by reference). It is contemplated that the methods of the present invention can be used to produce lymphoid, myeloid and erythroid cells from hematopoietic stem cells. The lymphoid lineage, comprising B-cells and T-cells, provides for the production of antibodies, regulation of the cellular immune system, detection of foreign agents in the blood, detection of cells foreign to the host, and the like. The myeloid lineage, which includes monocytes, granulocytes, megakaryocytes as well as other cells, monitors for the presence of foreign bodies in the blood stream, provides protection against neoplastic cells, scavenges foreign materials in the blood stream, produces platelets, and the like. The erythroid lineage provides the red blood cells, which act as oxygen carriers.

Accordingly, the present invention also contemplates the use of neural stem cells, which are generally isolated from developing fetuses. The isolation, culture, and use of neural stem cells are described in U.S. Pat. Nos. 5,654,183; 5,672,499; 5,750,376; 5,849,553; and 5,968,829, all of which are incorporated herein by reference. It is contemplated that the methods of the present invention can use neural stem cells to produce neurons, glia, melanocytes, cartilage and connective tissue of the head and neck, stroma of various secretory glands and cells in the outflow tract of the heart.

In other embodiments, the nanogel composition is loaded into immune system cells. The immune system cells can be derived from either passive or active immune systems. In some embodiments, the passive immune system cells are leukocytes, for example, neutrophils, macrophages, dendritic cells, mast cells, eosinophils, basophils, monocytes and natural killer cells. In some embodiments, the active immune system cells are lymphocytes, for example, T cells, killer T cells, and B cells.

In still further embodiments, the cells can be any cell that is known to home to a particular site within the body such mesenchymal cells, endothelial cells, neural cells, etc.

### 2. Nanogels

In some embodiments, the present invention provides nanogels comprising nanoparticles and optionally one or more active agents, labeling agents, and/or lytic agents. The active agents are described in more detail below and are preferably agents that are biologically active in the body, for example, a therapeutic compound, and chemotherapeutic compounds, a therapeutic protein, an antibiotic compound or an antiviral compound. In some embodiments, the active agent is encapsulated by the nanoparticle, attached to the nanoparticle or nanogel composition, adsorbed to the nanoparticle or nanogel composition, or otherwise associated with the nanoparticle or nanogel composition.

In some embodiments, the nanogel comprises nanoparticles formed from one or more polymeric materials. In some embodiments, the nanoparticles comprise one or more homopolymers, copolymers, random polymers, graft polymers, alternating polymers, block polymers, branch polymers, arborescent polymers or dendritic polymers or combinations thereof. Specific examples of polymers of use in the present invention include, but are not limited to, polyethylene glycol (PEG), polyethylenimine (PEI), polyglycolic acid (PGA), polylactic acid (PLA), N-isopropylacrylamide, acrylic acid, polypropylene glycol), poly(vinyl methyl ether), poly(N-isopropyl acrylamide), methacrylic acid, Et acrylate, N-isopropylmethacrylamide, poly(N-vinyl formamide), polyvinylamine, cholesteryl pullulan, Poly(DL-lactic-co-glycolic acid) and the like. In some embodiments, the nanogel composition is a copolymer formed by cross-linking two or more of the foregoing polymers, for example, PEG-PEI, N-isopropylacrylamide and acrylic acid, PG and PLA, methacrylic acid and Et acrylate, N-isopropylmethacrylamide and acrylic acid, poly(N-vinyl formamide) and polyvinylamine. In other embodiments, the nanoparticles are formed by coating one polymer, e.g., PLGA, with another, e.g., polyvinylamine. In preferred embodiments, the nanogel is nontoxic. In some preferred embodiments, the nontoxic nanogel is formed from PEG and PEI. Preferably, the methylene proton ratio (CH₂O:CH₂N) of the PEG-PEI nanogel is from about 6.0:1 to about 8.0:1, and most preferably about 6.8:1.

In some embodiments, the nanoparticles making up the nanogel are at least 0.1 nm, 1 nm, 10 nm, 20 nm, 30 nm, 40 nm, 50 nm, 60 nm, 70 nm, 80 nm, 90 nm or 100 nm in average diameter and less than 150 nm, 200 nm, 300 nm, 400 nm or 500 nm in average diameter. In some preferred embodiments, the nanoparticles making up the nanogel are from about 1 nm 10 to 100, preferably from about 5 nm to about 75 nm, more preferably about 10 nm to about 60 nm and most preferably about 20 nm to about 50 nm in diameter, or the largest dimension. For example, nanoparticles that are approximately 25 nm in diameter (the largest dimension) may be from 0.1 to 10 nm, or from about 1 to 5 nm in width.

### 3. Active Agents

In some preferred embodiments of the present invention, the nanogels described above comprise one or more active agents. In some embodiments, the active agent is a therapeutic compound, therapeutic protein, antibiotic, antiviral, or chemotherapeutic agent. In some embodiments, the active agent is not a nucleic acid, i.e., a non-nucleic acid active agent. In some embodiments, the active agent incorporated into the nanogel is present in a therapeutically effective amount, or that amount of the active agent that is required to produce a biological effect at the site of delivery of the nanogel.

In some embodiments, the active agent is a therapeutic compound. In some embodiments, the therapeutic compounds are small molecule drugs. In some embodiments, the therapeutic compound is insoluble under physiological conditions. In some embodiments, the therapeutic compound is a chemotherapeutic compound used to destroy or otherwise prevent the growth of tumor and/or cancer cells.

Examples of chemotherapeutic compounds include, but are not limited to, AQ10 (6-(hydroxymethyl)-1,4-anthracenedione), Methotrexate, Paclitaxel, Doxorubicin Hydrochloride, Fluorouracil, Imiquimod, Pemetrexed Disodium, Aminolevulinic Acid, Anastrozole, Aprepitant, Anastrozole, Exemestane, Nelarabine, Arsenic Trioxide, Azacitidine, Bendamustine Hydrochloride, Bexarotene, Bortezomib, Irinotecan Hydrochloride, Capecitabine, Carboplatin, Cetuximab, Cisplatin, Cyclophosphamide, Clofarabine, Clofarabine, Clofarabine, Cyclophosphamide, Cytarabine, Cytarabine, Cyclophosphamide, Decitabine, Dasatinib, Decitabine, Liposomal Cytarabine), Liposomal Cytarabine, Dexrazoxane Hydrochloride, Docetaxel, Doxorubicin Hydrochloride, Fluorouracil, Leuprolide Acetate, Epirubicin Hydrochloride, Oxaliplatin, Aprepitant, Epirubicin Hydrochloride, Erlotinib Hydrochloride, Raloxifene Hydrochloride, Exemestane, Fulvestrant, Letrozole), Gefitinib, Gemcitabine Hydrochloride, Imatinib Mesylate, Topotecan Hydrochloride, Imiquod, Gefitinib, Irinotecan Hydrochloride, Ixabepilone, Palifermin, Lapatinib Ditosylate Lenalidomide, Letrozole, Leuprolide Acetate, Aminolevulinic Acid, Nelarabine, Cyclophosphamide, Sorafenib Tosylate, Nilotinib, Tamoxifen Citrate, Pegaspargase, Palifermin, Carboplatin, Pemetrexed Disodium, Lenalidomide, Sorafenib Tosylate, Dasatinib, Sunitinib Malate, Thalidomide, Erlotinib Hydrochloride, Bexarotene, Nilotinib, Docetaxel, Temozolomide, Temsirolimus, Dexrazoxane Hydrochloride, Topotecan Hydrochloride, Bendamustine Hydrochloride, Arsenic Trioxide, Lapatinib Ditosylate, Bortezomib, Capecitabine, Dexrazoxane Hydrochloride, Zoledronic Acid, and Vorinostat.

In some embodiments, the therapeutic compound is a small molecule drug. Examples of small molecule drugs include, but are not limited to: ACE inhibitors, actin inhibitors, analgesics, anesthetics, anti-hypertensives, anti polymerases, antisecretory agents, anti-AIDS substances, antibiotics, anti-cancer substances, anti-cholinergics, anti-coagulants, anti-convulsants, anti-depressants, anti-emetics, antifungals, anti-glaucoma solutes, antihistamines, antihypertensive agents, anti-inflammatory agents (such as NSAIDs), Cox-2 inhibitors, antimetabolites, antimitotics, antioxidizing agents, anti-parasite and/or anti-Parkinson substances, antiproliferatives (including antiangiogenesis agents), anti-protozoal solutes, anti-psychotic substances, anti-pyretics, antiseptics, anti-spasmodics, antiviral agents, calcium channel blockers, cell response modifiers, chelators, chemotherapeutic agents, dopamine agonists, extracellular matrix components, fibrinolytic agents, free radical scavengers, growth hormone antagonists, hypnotics, immunosuppressive agents, immunotoxins, inhibitors of surface glycoprotein receptors, microtubule inhibitors, miotics, muscle contractants, muscle relaxants, neurotoxins, neurotransmitters, opioids, photodynamic therapy agents, prostaglandins, remodeling inhibitors, statins, steroids, thrombolytic agents, tranquilizers, vasodilators, and vasospasm inhibitors; enzyme inhibitors such as edrophonium chloride, N-methylphysostigmine, neostigmine bromide, physostigmine sulfate, tacrine HCl, tacrine, 1-hydroxymaleate, iodotubercidin, p-bromotetramisole, 10-(alpha-diethylaminopropionyl)-phenothiazine hydrochloride, calmidazolium chloride, hemicholinium-3,3,5-dinitrocatechol, diacylglycerol kinase inhibitor I, diacylglycerol kinase inhibitor II, 3-phenylpropargylamine, N-monomethyl-L-arginine acetate, carbidopa, 3-hydroxybenzylhydrazine HCl, hydralazine HCl, clorgyline HCl, deprenyl HCl, L(−), deprenyl HCl, D(+), hydroxylamine HCl, iproniazid phosphate, 6-MeO-tetrahydro-9H-pyrido-indole, nialamide, pargyline HCl, quinacrine HCl, semicarbazide HCl, tranylcypromine HCl, N,N-diethylaminoethyl-2,2-diphenylvalerate hydrochloride, 3-isobutyl-1-methylxanthine, papaverine HCl, indomethacin, 2-cyclooctyl-2-hydroxyethylamine hydrochloride, 2,3-dichloro-.alpha.-methylbenzylamine (DCMB), 8,9-dichloro-2,3,4,5-tetrahydro-1H-2-benzazepine hydrochloride, p-aminoglutethimide, p-aminoglutethimide tartrate, R(+), p-aminoglutethimide tartrate, S(−), 3-iodotyrosine, alpha-methyltyrosine, L(−) alpha-methyltyrosine, D L(−), cetazolamide, dichlorphenamide, 6-hydroxy-2-benzothiazolesulfonamide, and allopurinol; statins such lovastatin, pravastatin, simvastatin, fluvastatin, atorvastatin, cerivastatin, rousvastatin, and superstatin; cyclosporine, everolimus, mycophenolic acid, sirolimus, tacrolimus, and the like.

In some embodiments, the small drug compound is an anti-inflammatory agent. Examples of anti-inflammatory agents include, but are not limited to, diclofenac, etoldolac, fenoprofen, floctafenine, flurbiprofen, ibuprofen, indoprofen, ketoprofen, ketorolac, lomoxicam, morazone, naproxen, perisoxal, pirprofen, pranoprofen, suprofen, suxibuzone, tropesin, ximoprofen, zaltoprofen, zileuton, and zomepirac, and analogs, derivatives, pharmaceutically acceptable salts, esters, prodrugs, codrugs, and protected forms thereof; desmorphine, dezocine, dihydromorphine, eptazocine, ethylmorphine, glafenine, hydromorphone, isoladol, ketobenidone, p-lactophetide, levorphanol, moptazinol, metazocin, metopon, morphine, nalbuphine, nalmefene, nalorphine, naloxone, norlevorphanol, normorphine, oxmorphone, pentazocine, phenperidine, phenylramidol, tramadol, and viminol, and analogs, derivatives, pharmaceutically acceptable salts, esters, prodrugs, codrugs, and protected forms thereof; 21-acetoxypregnenolone, alclometasone, algestone, anacortave acetate, amcinonide, beclomethasone, betamethasone, budesonide, chloroprednisone, clobetasol, clobetasone, clocortolone, cloprednol, corticosterone, cortisone, cortivazol, deflazacort, desonide, desoximetasone, diflorasone, diflucortolone, difuprednate, enoxolone, fluazacort, flucloronide, flumethasone, flunisolide, fluocinolone acetonide, fluocinonide, flucloronide, flumethasone, flunisolide, fluocortin butyl, fluocortolone, fluorometholone, fluperolone acetate, fluprednisolone, flurandrenolide, fluticasone propionate, hydrocortamate, hydrocortisone, meprednisone, methylprednisolone, paramethasone, prednisolone, prednisolone 21-diethylaminoacetate, fluprednidene acetate, formocortal, loteprednol etabonate, medrysone, mometasone furoate, prednicarbate, prednisolone, prednisolone 25-diethylaminoacetate, prednisolone sodium phosphate, prednisone, prednival, prednylidene, triamcinolone, triamcinolone acetonide, triamcinolone benetonide, and triamcinolone hexacetonide, and analogs, derivatives, pharmaceutically acceptable salts, esters, prodrugs, codrugs, and protected forms thereof.

In some embodiments, the active agent is an antibiotic compound. Examples of antibiotic compounds useful in the present invention include, but are not limited to, capreomycins, including capreomycin IA, capreomycin IB, capreomycin IIA and capreomycin IIB; carbomycins, including carbomycin A; carumonam; cefaclor, cefadroxil, cefamandole, cefatrizine, cefazedone, cefazolin, cefbuperazone, cefcapene pivoxil, cefclidin, cefdinir, cefditoren, cefime, ceftamet, cefinenoxime, cefinetzole, cefminox, cefodizime, cefonicid, cefoperazone, ceforanide, cefotaxime, cefotetan, cefotiam, cefoxitin, cefpimizole, cefpiramide, cefpirome, cefprozil, cefroxadine, cefsulodin, ceftazidime, cefteram, ceftezole, ceftibuten, ceftiofur, ceftizoxime, ceftriaxone, cefuroxime, cefuzonam, cephalexin, cephalogycin, cephaloridine, cephalosporin C, cephalothin, cephapirin, cephamycins, such as cephamycin C, cephradine, chlortetracycline; chlarithromycin, clindamycin, clometocillin, clomocycline, cloxacillin, cyclacillin, danofloxacin, demeclocyclin, destomycin A, dicloxacillin, dicloxacillin, dirithromycin, doxycyclin, epicillin, erythromycin A, ethanbutol, fenbenicillin, flomoxef, florfenicol, floxacillin, flumequine, fortimicin A, fortimicin B, forfomycin, foraltadone, fusidic acid, gentamycin, glyconiazide, guamecycline, hetacillin, idarubicin, imipenem, isepamicin, josamycin, kanamycin, leumycins such as leumycin A₁, lincomycin, lomefloxacin, loracarbef, lymecycline, meropenam, metampicillin, methacycline, methicillin, mezlocillin, micronaomicin, midecamycins such as midecamycin A₁, mikamycin, minocycline, mitomycins such as mitomycin C, moxalactam, mupirocin, nafcillin, netilicin, norcardians such as norcardian A, oleandomycin, oxytetracycline, panipenam, pazufloxacin, penamecillin, penicillins such as penicillin G, penicillin N and penicillin O, penillic acid, pentylpenicillin, peplomycin, phenethicillin, pipacyclin, piperacilin, pirlimycin, pivampicillin, pivcefalexin, porfiromycin, propiallin, quinacillin, ribostamycin, rifabutin, rifamide, rifampin, rifamycin SV, rifapentine, rifaximin, ritipenem, rekitamycin, rolitetracycline, rosaramicin, roxithromycin, sancycline, sisomicin, sparfloxacin, spectinomycin, streptozocin, sulbenicillin, sultamicillin, talampicillin, teicoplanin, temocillin, tetracyclin, thostrepton, tiamulin, ticarcillin, tigemonam, tilmicosin, tobramycin, tropospectromycin, trovafloxacin, tylosin, and vancomycin, and analogs, derivatives, pharmaceutically acceptable salts, esters, prodrugs, codrugs, and protected forms thereof.

In some embodiments, the active agent is an antiviral compound. Anti-viral compounds are substances capable of destroying or suppressing the replication of viruses. Examples of anti-viral agents include neveripine, azidouridine, anasmycin, amantadine, bromovinyldeoxusidine, chlorovinyldeoxusidine, cytarbine, didanosine, deoxynojirimycin, dideoxycitidine, dideoxyinosine, dideoxynucleoside, desciclovir, deoxyacyclovir, edoxuidine, enviroxime, fiacitabine, foscamet, fialuridine, fluorothymidine, floxuridine, hypericin, interferon, interleukin, isethionate, nevirapine, pentamidine, ribavirin, rimantadine, stavirdine, sargramostin, suramin, trichosanthin, tribromothymidine, trichlorothymidine, vidarabine, zidoviridine, zalcitabine, 3-azido-3-deoxythymidine, 2′,3′-dideoxyadenosine (ddA), 2′,3′-dideoxyguanosine (ddG), 2′,3′-dideoxycytidine (ddC), 2′,3′-dideoxythymidine (ddT), 2′,3′-dideoxy-dideoxythymidine (d4T), 2′-deoxy-3′-thia-cytosine (3TC or lamivudime), 2′,3′-dideoxy-2′-fluoroadenosine, 2′,3′-dideoxy-2′-fluoroinosine, 2′,3′-dideoxy-2′-fluorothymidine, 2′,3′-dideoxy-2′-fluorocytosine, 2′,3′-dideoxy-2′,3′-didehydro-2′-fluorothymidine (Fd4T), 2′,3′-dideoxy-2′-beta-fluoroadenosine (F-ddA), 2′,3′-dideoxy-2′-beta-fluoro-inosine (F-ddI), and 2′,3′-dideoxy-2′-beta-fluorocytosine (F-ddC), acyclovir, azidouridine, anasmycin, amantadine, bromovinyldeoxusidine, chlorovinyldeoxusidine, cytarbine, didanosine, deoxynojirimycin, dideoxycitidine, dideoxyinosine, dideoxynucleoside, desciclovir, deoxyacyclovir, edoxuidine, enviroxime, fiacitabine, foscamet, fialuridine, fluorothymidine, floxuridine, ganciclovir, hypericin, interferon, interleukin, isethionate, idoxuridine, nevirapine, pentamidine, ribavirin, rimantadine, stavirdine, sargramostin, suramin, trichosanthin, trifluorothymidine, tribromothymidine, trichlorothymidine, trisodium phosphomonoformate, vidarabine, zidoviridine, zalcitabine and 3-azido-3-deoxythymidine, 3′ azido-3′ thymidine (AZT), dideoxyinosine (ddI), 2′,3′-dideoxyadenosine (ddA), 2′,3′-dideoxyguanosine (ddG), 2′,3′-dideoxycytidine (ddC), 2′,3′-dideoxythymidine (ddT), 2′,3′-dideoxy-dideoxythymidine (d4T), and 2′-deoxy-3′-thia-cytosine (3TC or lamivudime). Halogenated nucleoside derivatives may also be used including, for example, 2′,3′-dideoxy-2′-fluoronucleosides such as 2′,3′-dideoxy-2′-fluoroadenosine, 2′,3′-dideoxy-2′-fluoroinosine, 2′,3′-dideoxy-2′-fluorothymidine, 2′,3′-dideoxy-2′-fluorocytosine, and 2′,3′-dideoxy-2′,3′-didehydro-2′-fluoronucleosides including, but not limited to 2′,3′-dideoxy-2′,3′-didehydro-2′-fluorothymidine (Fd4T), 2′,3′-dideoxy-2′-beta-fluoroadenosine (F-ddA), 2′,3′-dideoxy-2′-beta-fluoro-inosine (F-ddI) and 2′,3′-dideoxy-2′-beta-fluorocytosine (F-ddC).

In some embodiments, the active agent is a therapeutic protein. Examples of therapeutic proteins include, but are not limited to, platelet-derived growth factor (pDGF), neutrophil-activating protein, monocyte chemoattractant protein, macrophage-inflammatory protein, SIS (small inducible secreted) proteins, platelet factor, platelet basic protein, melanoma growth stimulating activity, epidermal growth factor, transforming growth factor (alpha), fibroblast growth factor, platelet-derived endothelial cell growth factor, insulin-like growth factor, nerve growth factor, and bone growth/cartilage-inducing factor (alpha and beta), interleukins, interleukin inhibitors or interleukin receptors, including interleukin 1 through interleukin 10; interferons, including alpha, beta and gamma; hematopoietic factors, including erythropoietin, granulocyte colony stimulating factor, macrophage colony stimulating factor and granulocyte-macrophage colony stimulating factor; tumor necrosis factors, including alpha and beta; transforming growth factors (beta), including beta-1, beta-2, beta-3, inhibin, activin, heparin, heparin derivatives, sodium heparin, low molecular weight heparin, hirudin, lysine, prostaglandins, argatroban, forskolin, vapiprost, prostacyclin and prostacyclin analogs, D-ph-pr-arg-chloromethylketone (synthetic antithrombin), dipyridamole, glycoprotein IIb/IIIa platelet membrane receptor antibody, coprotein IIb/IIIa platelet membrane receptor antibody, recombinant hirudin, thrombin inhibitor (such as commercially available from Biogen), chondroitin sulfate, modified dextran, albumin, streptokinase, tissue plasminogen activator (TPA), urokinase, nitric oxide inhibitors, and the like, antibodies and antibody fragments including Abciximab, Alemtuzumab, Bevacizumab, Tositumomab and I 131 Iodine Tositumomab, Alemtuzumab, Bevacizumab, Cetuximab, Gemtuzumab Ozogamicin, Trastuzumab, Ibritumomab Tiuxetan, Panitumumab, Rituximab, Panitumumab, and Ibritumomab Tiuxetan and synthetic peptides or peptidomimetics such as eptifibatide or tirofiban.

In some embodiments, the therapeutic protein is a peptide. In some preferred embodiments, the peptide is an antimicrobial polypeptide such as tachyplesin I or II. Other examples of suitable antimicrobial polypeptides include, but are not limited to, human beta-defensins 1, 2, and 3, cathelicidin, LL37, magainin, buforin I, buforin II, indolicidin, nisin, cecropin A, B or C, ranalexin, lactoferricin B, dermaseptin 1, 2 or 3, bactenecin, BNP-1, HNP 1, 2, 3 or 4, neutrophil defensin 1 or 2, etc.

In some embodiments, the active agent is a protease inhibitor. One or more proteinases can preferably be included. Examples of protease inhibitors include, but are not limited to, aprotinin, bestatin, leupeptin, E-64 and pepstatin A and combinations thereof which inhibit serine, cysteine, aspartic and aminopeptideases. In further embodiments, metal chelators (e.g., EDTA) are included with the protease inhibitors. Non-peptide protease inhibitors may also be included, for example, 4-(2-aminoethyl)benzenesulfonyl flouride (AEBSH). By incorporating one or more of these protease inhibitors, different families of proteases in a cancer cell can be inhibited. For example, MMP2 and MMP7 proteases allow cancer cells to penetrate basal laminae as the first step in invasion and metastasis Inhibition of the proteases may slow these processes.

Other active agents that can be used for altering gene function include plasmids, phages, cosmids, episomes, and integratable DNA fragments, antisense oligonucleotides, antisense DNA and RNA, modified DNA and RNA, iRNA, ribozymes, siRNA, and shRNA.

### 4. Lytic Agents

In some embodiments, the cell and nanogel composition of the present invention further comprise a lytic agent. In some preferred embodiments, the lytic agent is incorporated into the nanogel composition. In some preferred embodiments, the lytic agent causes cell lysis or triggers apoptosis and subsequent cell lysis. In some embodiments, the lytic agent is provided in a concentration sufficient to cause cell lysis in a majority of the cells comprising the nanogel after a predetermined period of time, for example from 12 hours, 18 hours or 24 hours to about 48 hours, 72 hours, 96 hours, 120 hours or 240 hours.

In some embodiments, the lytic agent is a surfactant, preferably a detergent. In certain embodiments of the invention, the surfactant belongs to the TRITON™ X group of surfactants. TRITON™ X surfactants are versatile nonionic surfactants recognized for their wetting, detergency, superior hard surface, metal cleaning and excellent emulsification performance. In one illustrative embodiment, the nonionic surfactant is Triton X-100 which is also known as alkylaryl polyether alcohol; Octyl phenol ethoxylate; Polyoxyethylated octyl phenol; alpha-[4-(1,1,3,3-tetramethylbutyl)phenyl]-omega-hydroxypoly(oxy-1,2-etha-nediyl); Octoxynol; Triton X 100; Triton X 102; Ethylene glycol octyl phenyl ether; Polyoxyethylene octyl phenyl ether; p-(1,1,3,3-Tetramethylbutyl)phenol ethoxylate; Octylphenoxypolyethoxyethanol; Polyethylene glycol mono [4-(1,1,3,3-tetramethylbutyl)phenyl]ether; Poly(oxyethylene)-p-tert-octylphenyl ether; POE octylphenol; polyoxyethylene (10) octylphenol; POE (10) octylphenol; POE (10) Octyl Phenyl Ether; Octoxynol-10; POE (3) Octyl Phenyl Ether; Octoxynol-3; POE (30) Octyl Phenyl Ether; Octoxynol-30. The formula for Triton X-100 is C₁₄H₂₂O(C₂H₄₀)ₙ where the average number of ethylene oxide units per molecule is around 9 or 10. In another illustrative embodiment, the nonionic surfactant is Triton X-405, also known as 4-Octylphenol polyethoxylate, Poly(oxy-1,2-ethanediyl), alpha-(4-octylphenyl)-omega-hydroxy. In another illustrative embodiment, the nonionic surfactant is Triton BRIJ-35, also known as Polyoxyethylene monolauryl ether. In certain embodiments of the invention, the nonionic surfactant belongs to the Tween™ Series surfactants. In one such embodiment, the nonionic surfactant is Tween-20™ (C₅₈H₁₁₄O₂₆), also known as sorbitan mono-octadecanoate poly(oxy-1,1-ethanedlyl), polyoxyethylene sorbitan monolaurate, poly(oxyethylene) sorbitan monolaurate, polyoxyethylene (20) sorbitan monolaurate, Poe 20 sorbitan monolaurate, PSML, armotan pml-20, capmul, emsorb 6915, glycospere L-20 or liposorb L-20. In another such embodiment, the nonionic surfactant is Tween-80™, also known as polyethylene 20 sorbitan monooleate.

In certain embodiments of the invention, surfactant is a poloxamer. The term “poloxamer” is used according to its art accepted meaning and refers to any of a series of nonionic surfactants of the polyoxypropylene-polyoxyethylene copolymer type, having the general formula HO(C₂H₄O)ₐ(C₃H₆O)_{b}—(C₂H₄O)_{f}—H, where a=c; the molecular weights of the members of the series vary from about 1000 to more than 16,000. The term is used in conjunction with a numerical suffix for individual unique identification of products that may be used as a food, drug, or cosmetic. Poloxamers may be surfactants, emulsifiers, or stabilizers. In one such illustrative embodiment, the poloxamer is poloxamer 171.

In some embodiments, the lytic agent is a prodrug and is provided in combination with a suicide gene. In these embodiments, the selected cell line or population of cells is engineered to express the suicide gene, which encodes an enzyme that converts the prodrug into an active drug which causes apoptosis. In these embodiments, the cells are administered to a subject and allowed to migrate for a predetermined period of time, for example, 12, 14, 36, 48, 72, 96 120, or 240 hours. The prodrug is subsequently administered and is converted into the active drug by the cells.

In preferred embodiments, the suicide gene may be incorporated into a vector and introduced into the cell line or population of cells by methods known in the art. Vectors of the present invention preferably comprise a chemically synthesized or recombinant DNA molecule containing at least one suicide gene and appropriate nucleic acid sequences necessary for the expression of the operably linked coding sequence for the suicide gene, either in vitro or in vivo. Expression in vitro includes expression in transcription systems and in transcription/translation systems. Expression in vivo includes expression in a particular host cell and/or organism. Eukaryotic in vitro transcription systems and cells are known to utilize promoters, enhancers, and termination and polyadenylation signals. In an expression system suitable for expression in a eukaryotic cell, the promoter may be constitutive or inducible; the promoter may also be tissue or organ specific, or specific to a developmental phase. Preferably, the promoter is positioned 5′ to the transcribed region. Other promoters are also contemplated; such promoters include other polymerase III promoters and microRNA promoters. Preferably, a eukaryotic vector further comprises a transcription termination signal suitable for use with the promoter; for example, when the promoter is recognized by RNA polymerase III, the termination signal is an RNA polymerase III termination signal. The vector may also include sites for stable integration into a host cell genome.

Vectors may further comprise marker genes, reporter genes, selection genes, or genes of interest, such as experimental genes. Vectors of the present invention include cloning vectors and expression vectors; expression vectors are used in in vitro transcription/translation systems, as well as in in vivo in a host cell. Expression vectors used in vivo in a host cell are transfected into a host cell, either transiently, or stably. Thus, a vector may also include sites for stable integration into a host cell genome.

In some embodiments of the present invention, vectors include, but are not limited to, chromosomal, nonchromosomal and synthetic DNA sequences (e.g., derivatives of viral DNA such as vaccinia, adenovirus, fowl pox virus, and pseudorabies). It is contemplated that any vector may be used as long as it is expressed in the appropriate system (either in vitro or in vivo) and viable in the host when used in vivo; these two criteria are sufficient for transient transfection. For stable transfection, the vector is also replicable in the host.

Large numbers of suitable vectors are known to those of skill in the art, and are commercially available. In some embodiments of the present invention, mammalian expression vectors comprise an origin of replication, suitable promoters and enhancers, and also any necessary ribosome binding sites, polyadenylation sites, splice donor and acceptor sites, transcriptional termination sequences, and 5′ flanking non-transcribed sequences. In other embodiments, DNA sequences derived from the SV40 splice, and polyadenylation sites may be used to provide the required non-transcribed genetic elements. Promoters useful in the present invention include, but are not limited to, the cytomegalovirus (CMV) immediate early, herpes simplex virus (HSV) thymidine kinase, and mouse metallothionein-I promoters and other promoters known to control expression of gene in mammalian cells or their viruses. In other embodiments of the present invention, recombinant expression vectors include origins of replication and selectable markers permitting transformation of the host cell (e.g., dihydrofolate reductase or neomycin resistance for eukaryotic cell culture). In some embodiments of the present invention, transcription of DNA encoding a gene is increased by inserting an enhancer sequence into the vector. Enhancers are cis-acting elements of DNA, usually about from 10 to 300 by that act on a promoter to increase its transcription. Enhancers useful in the present invention include, but are not limited to, a cytomegalovirus early promoter enhancer, the polyoma enhancer on the late side of the replication origin, and adenovirus enhancers. In other embodiments, the expression vector also contains a ribosome binding site for translation initiation and a transcription terminator. In still other embodiments of the present invention, the vector may also include appropriate sequences for amplifying expression.

Exemplary vectors include, but are not limited to, the following eukaryotic vectors: pWLNEO, pSV2 CAT, pOG44, PXT1, pSG (Stratagene) pSVK3, pBPV, pMSG, pSVL (Pharmacia), and pCS2 vectors and its derivatives, as described in the Examples. Other plasmids are the Adenovirus vector (AAV; pCWRSV, Chatterjee et al. (1992) Science 258: 1485), a retroviral vector derived from MoMuLV (pG1Na, Zhou et al. (1994) Gene 149: 3-39), and pTZ18U (BioRad, Hercules, Calif., USA).

Suitable suicide gene/prodrug combinations include, but are not limited to, carboxylesterase gene/irinotecan; cytosine deaminase gene/5-fluorocytosine; carboxypeptidase G2 gene/(2-chloroethyl)(2-mesyloxyethyl)aminobenzoyl-L-glutamic acid; cytochrome p450 gene/cyclophosphamide, ifosfamide; ipomeanol, or 2-aminoanthracene; deoxycitidine kinase gene/cytosine arabinocide; HSV thymidine kinase gene/ganciclovir or acyclovir; ntrireductase gene/5-aziridinyl-2,4-dinitrobenzamide; purine nucleoside phosphorylase gene/6-methylpurine-2′-deoxyribonucleoside, thymidine phosphorylase gene/5′-deoxy-5-fluorouridine; vzv-thymidine kinase gene/6-methoxypurine arabinonucleoside; and xanthine-guanine phosphoribosyl transferase gene/6-thioxanthine or 6-thioguanine

### 5. Labeling Agents

In further embodiments, the nanogel compositions described above can optionally include a labeling agent. The labeling agent may be covalently or non-covalently attached to the nanogel composition. In some embodiments, the labeling agent is a fluorescent compound. Examples of suitable fluorescent compounds include, but are not limited to, Rodamine, Fluoroscein isothiocyanate (FITC), ALEXA 488, ALEXA 546, ALEXA 633, ALEXA 568, ALEXA 647, ALEXA 660, Cy2, Cy3, Cy3B, Cy5, Cy7, Sytox Blue, Cytox Green, Sytox Orange, Texas red, TAMARA, and TRITC. In other embodiments, the labeling agent is a fluorescent protein, for example, green fluorescent protein, yellow fluorescent protein, or red fluorescent protein. In still other embodiments, the labeling agent is a metallic nanoparticle, for example, nanogold particles.

### 6. Therapeutic Use

In some embodiments, the present invention provides methods for treating a subject (e.g., a human or animal), comprising administering to the subject a composition comprising cells that comprise a nanogel comprising an active agent. In some embodiments, the cells further comprise a lytic agent. In preferred embodiments, the cells are administered to the subject intravenously. The present invention is not limited to any particular mechanism of action. Indeed, an understanding of the mechanism of action is not necessary to practice the present invention. Nevertheless, it is contemplated that the cell compositions of the present invention are administered to a subject and subsequently deliver the nanogel comprising the active agent to a site within the body. In some embodiments, the cells are administered intravenously. It is contemplated that depending on the cell type chosen, particular areas or cell or tissue types within the body will be targeted, allowing targeting delivery of the active agent to a selected site within the body. In some embodiments, the cells preferably release the nanogel in the vicinity of the target and the active agent and/or nanogel are taken up by the targeted cells or tissue. In some preferred embodiments, the active agent destroys or otherwise inhibits the growth of cells in the target area, for example cancer cells or tumor cells. In some preferred embodiments, the tumor is breast cancer tumor or lung cancer tumor. In other preferred embodiments, the lytic agent causes lysis of the cells, preferably at the site of the targeted tissues or cells. In embodiments where the lytic agent is a surfactant, the surfactant included in the nanogel at a concentration that results in lysis of the cell within a predetermined time period that allows for migration of the cells. When the lytic agent is prodrug, the cells are allowed to migrate for a predetermined time and then the prodrug is administered. The prodrug triggers apoptosis of the cells at the targeted cells or tissue so that the active agent is delivered.

### EXPERIMENTAL

### Example 1

Different nanogels were synthesized with altered ratios of CH₂O:CH₂N (methylene proton) in PEG-PEI as determined by the ¹H NMR spectroscopy. Two nanogels with methylene proton ratios of 4:1 and ˜6.8:1 were used for in vitro testing on the mouse pancreatic adenocarcinoma cell line, Pan 02. The nanogels were labeled with rhodamine to enhance intracellular visualization. The nanogel with the methylene proton ratio of 4:1 was very toxic to Pan 02 cells while that with the methylene proton ratio of ˜6.8:1 was not, indicating that the methylene proton ratio is an important determinant of nanogel PEG-PEI toxicity. The size of the nontoxic nanogel was further characterized by PEG-PEI by AFM studies.

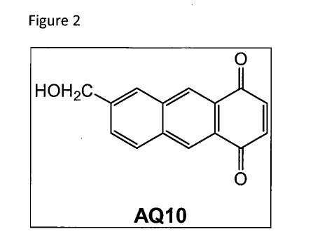

The AQ analogue, AQ10 (FIG. 2), first synthesized and characterized by Hua et. al. in 2006 was shown to significantly decrease HL-60 and LL/2 cancer cell growth by initially triggering early and late apoptosis, and later causing internucleosomal DNA fragmentation. AQ10 was incorporated into nanogel PEG-PEI with a methylene proton ratio of ˜6.8:1 and tested its effect on Pan 02 cell proliferation. The results showed that AQ10-nanogel PEG-PEI is significantly more effective in altering the growth of Pan 02 cells than AQ10 or nanogel PEG-PEI alone.

### Materials and Methods

Materials. PEI (˜25 kDa), PEG (8 kDa), 1,1′-carbonyldiimidazole, N,N′-dicyclohexylcarbodiimide (DCC), N-hydroxysuccinimide, and 1-hydroxybenzotriazole (HOBT) were purchased from Aldrich Chemical Co. AQ10 was prepared as described. [Perchellet et al., Biochem. Pharmacol., 67, 523 (2004); Hua et al., Anticancer Agents Med. Chem., 6, 303 (2006)]. Nanogel PEG-PEI was prepared by following a reported micellar method [Vinogradov et al., Pharm. Res., (2006)] with modification, and spectral characterization of the produced nanogel PEG-PEI was studied (vide infra). 6-Carboxytetramethylrhodamine (TAMRA) was purchased from Invitrogen and was activated with DCC, HOBT, and N-hydroxysuccinimide to product TAMRA-SE. Sephacryl S200 and membranes were purchased from Fisher Scientific. ¹H NMR spectra were recorded on 200 and 400 MHz Varian UNITYplus instruments. Atomic force microscopy (AFM) images were obtained from a Nanoscope IIIa SPM atomic force microscope (Digital Instruments, Inc. Santa Barbara, Calif.). A HP 8543 UV-Vis Spectrophotometer was used for obtaining UV-vis spectra. The murine Pan 02 cell line was obtained from DCTD Tumor Repository (NCI).

AFM experiments. AFM images were collected using a tapping mode with a high aspect ratio tip (Veeco Nanoprobe TM tips, Model TESP-HAR). A solution of AQ10-nanogel PEG-PEI was prepared as followed for AFM studies. A solution of 2.7 mg of nanogel PEG-PEI-AQ10 was dissolved in 500 μL of deionized water and diluted with 240 μL of DMSO. A small aliquot (20 μL) of each sample was removed and placed onto freshly cleaved mica, washed with deionized water twice, and dried with N₂. AFM images on different locations of the mica were then obtained from a Nanoscope Ma SPM instrument. Similarly, AFM images of a sample of PEG-PEI-rhodamine-1% AQ10 were also measured.

Separation of PEI. A Sephacryl S200 (30 g) chromatographic column was prepared with deionized water. To it was added 7.0 g (0.28 mmol) of PEI (MW ˜25 kDa; contaminated with lower and higher MW materials) in 20 ml of deionized water. Deionized water was used as eluant. The middle fractions (based on weight distribution) were collected and lyophilized to give 3.64 g (0.146 mmol) of PEI (MW ˜25 kDa). ¹H NMR (D₂O) δ 2.72 (bs, CH₂N), 2.68 (bs, CH₂N); the above two signals are overlapped and the number of hydrogens cannot be determined from integration.

Activation of PEG. To a solution of 2.0 g (0.25 mmol) of PEG (MW 8 kDa) in 7 ml of dry acetonitrile under argon was added 0.41 g (2.5 mmol) of 1,1′-carbonyldiimidazole, and the solution was stirred at 40° C. for 2 hours. The crude product was dialyzed twice using a MWCO 2 kDa membrane twice with 800 ml of 10% ethanol in deionized water at 4° C. for 4 hours. The solution was lyophilized to give 1.84 g of activated PEG. ¹H NMR (CDCl₃) δ 7.69 (s, 1H, ArH), 7.11 (s, 2H, ArH), 3.62 (s, 190H, CH₂O).

Preparation of nanogel PEG-PEI. Synthesis started from activated PEG and PEI (Scheme 1). To a solution of 1.0 g (40 μmol) of PEI (MW ˜25 kDa) in 300 ml of deionized water was added dropwise a solution of 0.50 g (62.5 μmol) of activated PEG (MW ˜8 kDa) in 2 ml of dichloromethane. The reaction solution was sonicated in a water bath for 10 minutes, and the organic solvent was removed on a rotary evaporator resulting in a transparent solution. The solution was dialyzed with a 12K-14K MWCO membrane in 800 ml of 10% ethanol in deionized water for 1 day at 25° C. and lyophilized to give nanogel PEG-PEI. This nanogel PEG-PEI was again treated with 1.0 g (125 μmol) of activated PEG in 2 ml of dichloromethane and worked up as mentioned above to give 1.32 g of nanogel PEG-PEI. ¹H NMR (D₂O) δ 3.70 (s, area 44, CH₂O), 3.40-2.60 (m, area 6.5, CH₂N). Based on the weight of the product, it is estimated that the molecular weight of the nanogel PEG-PEI is ˜33 KDa (for each mole of PEI, one mole of PEG is added). The initial treatment of PEI with activated PEG provided a partial cross-linkage of PEG, in which for each mole of PEI, there is ˜0.5 mole of PEG attached.

Synthesis of nanogel PEG-PEI-rhodamine. A mixture of 15 mg (32 μmol) of 6-carboxytetramethylrhodamine (TAMRA), 9.9 mg (48 μmol) of DCC, 6.52 mg (48 μmol) of HOBT, and 4.44 mg (39 μmol) of N-hydroxysuccinimide was dried under vacuum and maintained under argon. To it, 1 ml of dry DMF was added via syringe. The resulting solution was stirred at 50-55° C. for 2.5 hours, cooled to room temperature, and added a solution of 200 mg of nanogel PEG-PEI in 1 ml of acetonitrile. The solution was stirred at 40° C. for 12 hours, cooled to 25° C., dialyzed with a 12 k-14 k MWCO membrane in 10% ethanol in deionized water at room temperature for 1 day, and lyophilized to give 186 mg of PEG-PEI-rhodamine. ¹H NMR (D₂O) δ 8.50 (s, area 0.03), 8.10 (m, area 0.015), 7.90 (m, area 0.015), 7.73 (m, area 0.015), 7.37 (m, area 0.06), 3.70 (s, area 100, CH₂O), 3.20-2.60 (m, area 14.7,CH₂N). UV-vis (H₂O), λₘₐₓ=557 nm and εₘₐₓ=1.57×10⁴ M⁻¹·cm⁻¹ (assuming the MW ˜33 KDa)

Inclusion of AQ10 (1%) in nanogel PEG-PEI-rhodamine. To a solution of 50 mg of nanogel PEG-PEI-rhodamine in 5 ml of deionized water, 0.5 mg (2.1 μmol) of AQ10 in 1 ml of acetonitrile was added. The resulting solution was lyophilized to give 50.5 mg of nanogel PEG-PEI-rhodamine-AQ10.

Inclusion of AQ10 (5%) in nanogel PEG-PEI-rhodamine. To a solution of 50 mg of nanogel PEG-PEI-rhodamine in 5 ml of deionized water, 2.5 mg (10.5 μmol) of AQ10 in 1 ml of acetonitrile was added. The resulting solution was lyophilized to give 52.5 mg of nanogel PEG-PEI-rhodamine-AQ10. The 5% AQ10 has greater antitumor effect than 1% AQ10.

Cell culture. Pan 02 cells were maintained in medium containing RPMI 1640 (Invitrogen), 10% fetal bovine serum (FBS, Atlanta Biologicals), and 1× pen/strep (Invitrogen) at 37° C. in a humidified atmosphere containing 5% carbon dioxide.

Loading of nanogel PEG-PEI into Pan 02 cells. Pan 02 cells were seeded at 3×10⁴ in a 12-well plate. At ˜70% confluency, nanoparticles were added at 0.05 mg/well and incubated for 12 hrs. Following incubation, excess nanoparticles were removed by washing wells with 1× PBS, and fresh media was added. The loading of nanoparticles into cancer cells was visualized using a Nikon Eclipse epifluorescent microscope. Images were captured using a Roper Cool Snap ES camera and Metamorph 7 image analysis system.

Cell proliferation evaluation. The number of viable cells was evaluated by the MTT (3-[4,5-methylthiazol-2-yl]-2,5-diphenyl-tetrazolium bromide) assay (Roche Diagnostics GmbH, Germany) as well as hemocytometer and trypan blue exclusion analysis. Pan 02 cells were seeded in a 96 well plate. After reaching ˜70% confluency, the media was replaced with fresh medium containing DMSO or AQ10+DMSO, and nanogel PEG-PEI with different ratio's of methylene proton ratio or AQ10-nanogel PEG-PEI at different concentrations. After incubation for 48 hrs, the medium was replaced with fresh medium containing 0.5 mg of MTT reagent per ml of medium and incubated for four hours at 37° C. Then, 100 μL of solubilization reagent was added to each well and incubated overnight at 37° C. Absorbance was measured in an ELISA plate reader at 550 nm, with the absorbance at 690 nm to correct for background, and viability was expressed as the percentage of untreated controls. To validate the MTT assay, trypan blue exclusion and hemocytometer counting assay was performed in parallel. All experiments were done in duplicate and were performed three times.

Statistical analysis. All values are expressed as means ±SE. Values of untreated cells were taken as 0%. Statistical analysis of all Pan 02 cell proliferation assays were analyzed using repeated measures of ANOVA. When a significant F-ratio was demonstrated by the ANOVA-R, post hoc tests (Bonferroni) were applied to describe significant Pan 02 cell proliferation and dose effect of nanogel PEG-PEI AQ10, AQ10, DMSO. Statistical significance was considered if P<0.05.

### Results and Discussion

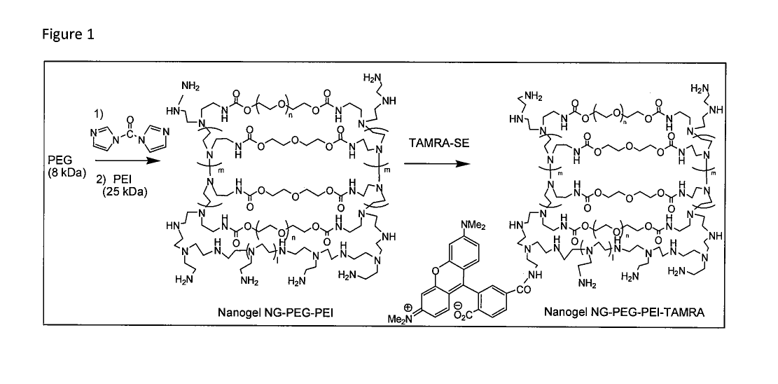

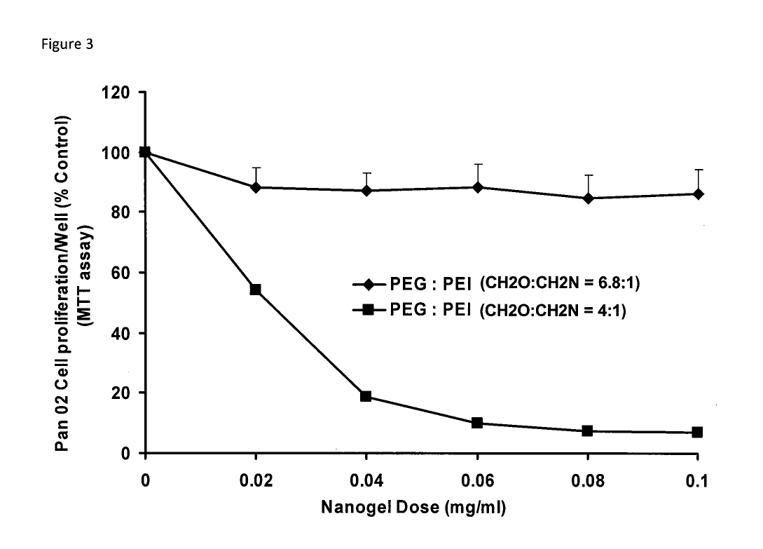

Structure, synthesis and toxicity of nanogel PEG-PEI and rhodamine-attached nanogel PEG-PEI. Based on the nanogel synthesis method, the putative nanogel PEG-PEI structure is shown in FIG. 1. However, this structure has not been confirmed; therefore, ¹H NMR spectrum was performed, which revealed a ratio of ˜6.8:1 for ¹H NMR (methylene proton ratio). Based on the weight of the product from the coupling reaction of PEG and PEI, the ratio of PEG: PEI is ˜1:1; this preparation of nanogel PEG-PEI was found to be non-toxic to cancer cells. However, when the methylene proton ratio was decreased to 4:1, the nanogel PEG-PEI was highly toxic to cancer cells (FIG. 3). This 4:1 ratio of nanogel PEG-PEI along with other different ratios of CH₂O to CH₂N in PEG-PEI was prepared from different treatments of activated PEG with PEI (see methods). Thus, an important finding was that the methylene proton ratio is critical and should be kept ≧˜6.8:1 this was achieved by repeated PEGylation of nanogel to avoid significant solubility issues or undesirable bioactivities, such as cytotoxicity. When studying the effects of incorporating small molecule drugs such as AQ10 into the nanogel PEG-PEI, it is important to limit nanogel PEG-PEI toxicity in order to distinguish anticancer effects of the small molecule drug from cytotoxic effects of the carrier. The attachment of rhodamine helps to study the incorporation of nanogel PEG-PEI into cells. ¹H NMR spectrum revealed that only a small amount of rhodamine molecules were attached to PEG-PEI complex. UV-vis spectrum of nanogel PEG-PEI-rhodamine showed the presence of rhodamine in the nanogel PEG-PEI.

Studies on the sizes of nanogel PEG-PEI-AQ10 particles by AFM. The sizes of nanogel PEG-PEI-AQ10 particles were measured using an AFM instrument with tapping mode. Several samples were prepared and they exhibited similar images. Some of the small nanogel PEG-PEI particles aggregate to form short fibril-like materials. Overall, the nanogel PEG-PEI-AQ10 particles were rather evenly sized, small, round particles with diameter of ˜23 nm and height of 1 nm (data not shown). Similarly, AFM images of PEG-PEI-rhodamine-1% AQ10 were also obtained and they are similar to the aforementioned nanogel without rhodamine.

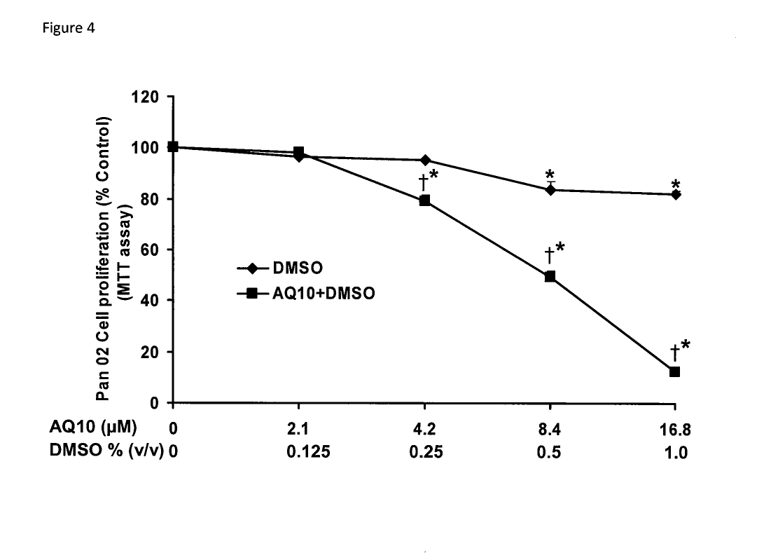

AQ10 inhibits Pan 02 Cell Viability in a dose dependent manner. Previous studies showed that AQ10 can significantly decrease HL-60 and LL/2 cancer cell growth. The effect of AQ10 alone was tested on Pan 02 cell proliferation. The results from the cell proliferation assay showed that, AQ10 in DMSO significantly attenuated Pan 02 cell growth at doses of 4.2, 8.4 and 16.8 μM compared to untreated and DMSO treated (0.125, 0.25, 0.5, 1% (v/v) cells (FIG. 4).

Uptake and cellular distribution of nanogel PEG-PEI in Pan 02 cells. The potential uptake of nanogel PEG-PEI by Pan 02 cells was tested. The AQ10-nanogel PEG-PEI was covalently tagged with rhodamine to visualize whether AQ10-nanogel PEG-PEI was internalized by Pan 02 cells. The majority of red-fluorescent labeled nanoparticles were distributed in the cytoplasm of Pan 02 cells over a period of 12 hours (data not shown).

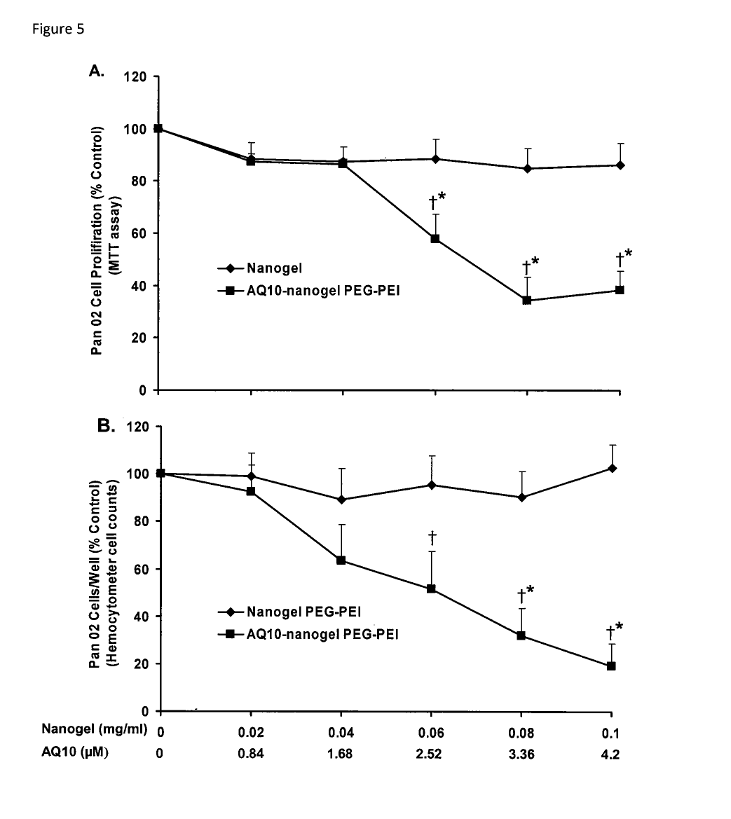

AQ10-nanogel PEG-PEI inhibited Pan 02 cell proliferation in a dose-dependent manner. The results from the MTT assay (FIG. 5A) showed that nanogel PEG-PEI (−6.8:1, methyl proton ratio) by itself had no significant effect on the Pan 02 cell viability compared to untreated cells. In contrast, incubation with 1% AQ10-nanogel PEG-PEI significantly decreased cell proliferation at doses of 0.06, 0.08 and 0.1 mg dose per ml of medium, compared to cells incubated with nanogel PEG-PEI alone and untreated cells. FIG. 5B shows the results from hemocytometer-trypan blue exclusion assays. A similar effect was noted: cells that were incubated with 1% AQ10-nanogel PEG-PEI showed a significant decrease in the total number of viable cells at doses of 0.06, 0.08, 0.1 mg dose per ml of medium compared to nanogel PEG-PEI alone. Importantly, AQ10 alone was three to four times less effective in growth attenuation of Pan 02 cells than AQ10 incorporated into nanogel PEG-PEI. Nanogel PEG-PEI complex has hydrophilic PEG that is exposed outside and hydrophobic PEI in the inside. Thus, it is likely that the drug, AQ10, is inside the nanogel via hydrophobic interactions between the drug and PEI. The present invention is not limited to any particular mechanism of action. Nevertheless, it is believed that after cellular uptake, AQ10 will be slowly released from the nanogel over a certain period of time due to interactions with other molecules inside the cells. This is presumably due to low water solubility of AQ10. Poor solubility of AQ10 in aqueous solution appears to make the drug less efficient; when incorporated into nanogel PEG-PEI, the AQ10 is more efficiently taken up by the cells, thereby decreasing the effective dose.

### CONCLUSION

This data provides experimental support for five new findings. First, the methylene proton ratio of PEG-PEI in the nanogel alters the toxicity of the nanogel PEG-PEI. Second, nanogel PEG-PEI can be loaded with and release a therapeutic anticancer drug, AQ10. Third, rhodamine (TAMRA) dye molecule can be incorporated into nanogel PEG-PEI to study the localization of nanogel PEG-PEI in cells. Fourth, AQ 10 dissolved in DMSO inhibits Pan 02 cell proliferation. Fifth, when AQ10 is incorporated into nanogel PEG-PEI, it causes a significant reduction in viable cell numbers of Pan 02 cells compared to AQ10 alone. These results suggest the possibility of using nanogel PEG-PEI as an efficient drug delivery vehicle by encapsulating cytotoxic anticancer compounds such as AQ10. In conclusion, these studies have shown that the nanogel PEG-PEI system can be used to deliver poorly soluble, toxic synthetic anticancer drugs for potential therapeutic application for pancreatic and other cancers.

### Example 2

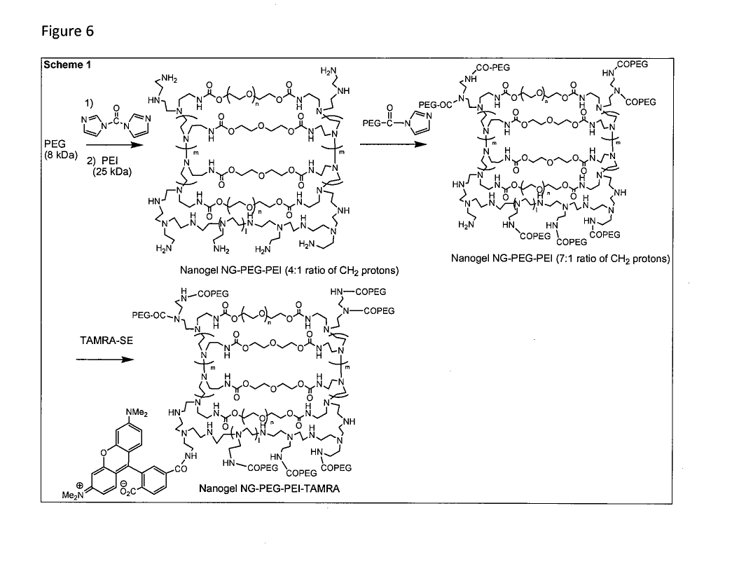

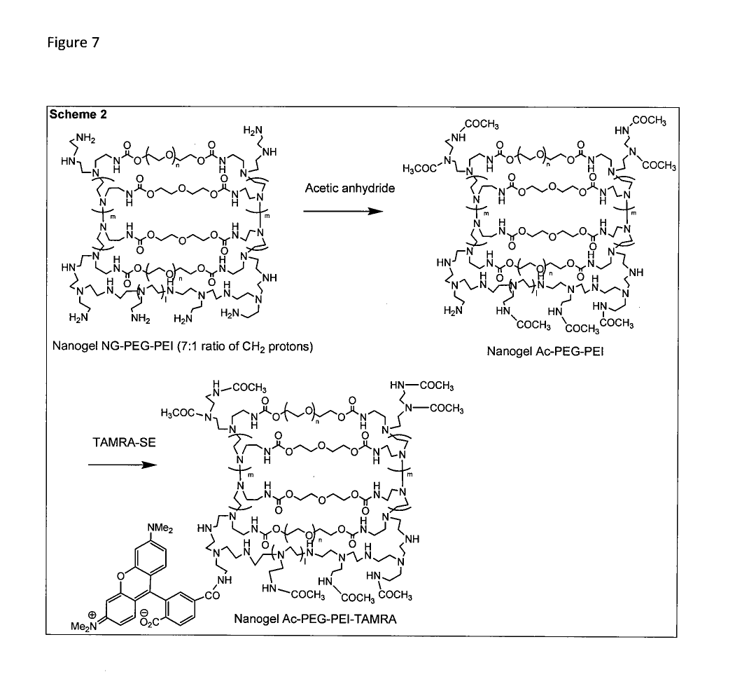

Novel non-toxic acetylated PEG-PEI nanogel was synthesized by an acetylation reaction of toxic PEG-PEI nanogel. Initially, the reported procedure [Vinogradovet al., Pharm. Res. 23, 920-930.] was followed to prepare PEG-PEI nanogel (FIG. 6). However, this nanogel is toxic to cells, including normal cells, cancer cells and stem cells. The ¹H NMR spectrum of this nanogel indicated a ratio of methylene protons of CH₂ groups of PEG and PEI is 4:1. This nanogel with another round of activated PEG to increase the content of PEG in the nanogel (or masked the toxic amino function of the nanogel) (FIG. 6). When the methylene proton ratio of PEG to PEI reaches ˜7:1 after a second treatment of nanogel with activated PEG, the nanogel indeed is non-toxic to cells (at lease for a period of four days). The procedure has been modified by treating the non-toxic 7:1 methylene proton ratio (PEG:PEI) of nanogel with acetic anhydride (FIG. 7). The acetylated nanogel (called Ac-PEG-PEI nanogel) is non-toxic to cells (for a period of four days). It should be noted that both double treated PEI with activated PEG and Ac-PEG-PEI nanogels are new compounds.

Beside acetic anhydride, other anhydrides are used to synthesize different nanogels with various alkyl, alkenyl, and aryl groups attached. These appendages allow the incorporation of various functionalities to alter the physical properties of the nanogels.

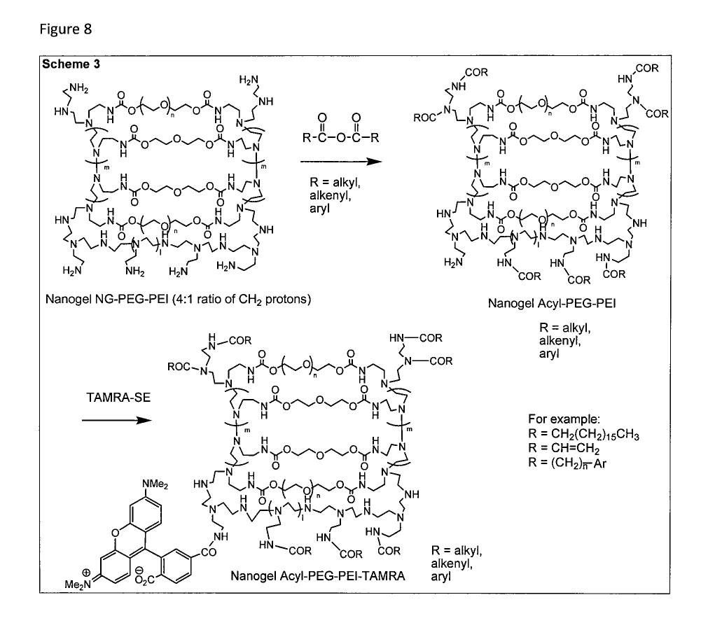

FIG. 8 illustrates two new alkyl (C18, from stearic acid anhydride) and alkenyl (from acrylic anhydride) attached nanogels. The alkyl attached nanogel can provide self-assembled nanogel with a discrete structure, while the alkenyl function allows an internal polymerization (using a free radical initiator to initiates the polymerization) to provide cross-linked nanogel.

Anticancer drugs such as AQ10, TT24, and Paclitaxel (a known anticancer drug) were encapsulated into PEG-PEI nanogel and these nanogel-drugs were loaded into stem cells, neutrophils, and lymphocytes separately. The nanogel-drug-cells are expected to home to cancer cells and to inhibit cell growth. Nanogel PEG-PEI was also used to encapsulate bioactive peptides such as tachyplesin (Journal of Biological Chemistry, 1988, 263, 16709-713), 17-residue antimicrobial peptide (H₂N—K—W—C—F—R—V—C—Y—R-G—I—C—Y—R—R—C—R—CONH₂) for antimicrobial usages.

Experimental Procedures:

Preparation of PEG:PEI (methylene proton ratio of 4:1) Nanogel: To a stirred solution of 400 mg of PEI in 100 ml of deionized water, a solution of 600 mg of activated PEG (PEG was activated with carbonyl diimidazole) in 2 ml of dichloromethane was added dropwise. The solution was then sonicated for 10 minutes on a sonicator. Dichloromethane was removed under a rotary evaporator. The nanogel solution was dialyzed in 1000 ml solution of 10% ethanol in deionized water for 24 hours at room temperature using MWCO 12 k-14 k membrane. The resulting solution of nanogel (free of low molecular PEG) was lyophilized on a freeze dry instrument to obtain a white powder product. ¹H NMR spectrum of this material in D₂O indicated the methylene proton ratio of the CH₂O to CH₂N groups is 4:1.

Preparation of acetylated PEG-PEI (Ac-PEG-PEI) nanogel: To a solution of 100 mg of the above PEG:PEI (4:1) nanogel in 1 ml of acetonitrile, 200 μl of acetic anhydride was added by syringe. The solution was stirred at 50° C. for 12 hours and dialyzed in 1000 ml of 10% ethanol in deionized water for 24 hours at room temperature using MWCO 12-14 k membrane. The resulting solution was lyophilized to give 100 mg of Ac-PEG-PEI. ¹H NMR spectrum (in D₂O) indicated the acetyl group was incorporated into the nanogel.

Preparation of Ac-PEG-PEI-Rhodamine (Ac-PEG-PEI-TAMARA): to a Solution of 550 mg of Ac-PEG-PEI in 12 ml of acetonitrile was added a solution of 300 μl of activated rhodamine solution (TAMRA-SE; see Chanran Ganta et al. J. Nanoscience and Nanotechnology, 2008, 8(5), 2334-2340). The solution was stirred at 40° C. for 12 hours, cooled to room temperature, and dialyzed with 1000 ml of 10% ethanol in deionized water for 24 hours at room temperature using MWCO 12 k-14 k membrane. The resulting solution was lyophilized to give 550 mg of the desired Ac-PEG-PEI-TAMRA product (as a pink colored powder). ¹H NMR spectrum and fluorescence spectrum indicated the presence of rhodamine dye in the nanogel. Addition of more TAMRA to the preparation is an alternate way to cover the amine groups.

Preparation of Ac-PEG-PEI-TAMRA-Triton (1% Triton): To engineer a chemical for destroying neutrophil or other delivery cell after homing to cancer cells, Triton-X was incorporated into the nanogel-drug material. In every instance tested thus far, the Triton has significantly potentiated the anti-cancer effect of the drug. To a solution of 20 mg of Ac-PEG-PEI-TAMRA in 8 ml of deionized water, was added a solution of 200 μl of 0.1% Triton in deionized water. The solution was stirred for 1 minute and then lyophilized in a freeze dry instrument to give a pink colored powder.

Preparation of Ac-PEG-PEI-TAMRA-Triton (5% Triton): To a solution of 20 mg of Ac-PEG-PEI-TAMRA in 8 ml of deionized water, was added 1 ml of 0.1% Triton in deionized water. After stirring the solution at room temperature for 1 min. the solution was lyophilized to give the desired nanogel-Triton powder as a pink colored solid.

Preparation of Ac-PEG-PEI-TAMRA-Triton(1%)-AQ10(5%): To a solution of 30 mg of Preparation of Ac-PEG-PEI-TAMRA (1%) in 8 ml of deionized water, were added a solution of 1.5 mg of AQ10 in 1 ml of acetonitrile and 300 μA of 0.1% Triton in deionized water. The solution was stirred for 1 min. and lyophilized to give a pink powder product.

Preparation of Ac-PEG-PEI-TAMRA-Triton(5%)-AQ10(5%): To a solution of 30 mg of Ac-PEG-PEI-TAMRA in 8 ml of deionized water were added a solution of 1.5 mg of AQ10 in 1 ml acetonitrile and 1.5 ml of 0.1% Triton in deionized water. The resulting solution was stirred for 1 min. and lyophilized to give a pink powder product.

Preparation of Ac-PEG-PEI-TAMRA-AQ10(5%): To a solution of 30 mg of Ac-PEG-PEI-TAMRA in 8 ml of deionized water, was added a solution of 1.5 mg of AQ10 in 1 ml of acetonitrile. The solution was stirred for 1 min. and lyophilized to give a pink powder product.

Encapsulation of Paclitaxel (5% by weight) with nanogel PEG-PEI-rhodamine: Nanogel PEG-PEI-rhodamine, 20 mg (MW ˜35500; 0.56 μmol), was dissolved in 10 mL of deionized water. To it, a solution of 1 mg (MW 854; 1.2 μmol) of Paclitaxel (or taxol) in 1 mL of acetonitrile was added. The resulting solution was mixed thoroughly and lyophilized on a freeze dry instrument to give 21 mg of powder, which is soluble in water.

Encapsulation of SN-38 (5% by weight) with nanogel PEG-PEI-rhodamine: Nanogel PEG-PEI-rhodamine, 20 mg (MW 35500; 0.56 μmol), was dissolved in 2 mL of deionized water. To it, a suspension of 1 mg (MW 392; 2.6 μmol) of SN-38 in 1 mL of acetonitrile and 0.5 mL of methanol was added (a suspension was resulted after sonication). The resulted suspension was lyophilized to give a powder, which was used in the bio-screening.

Encapsulation of SN-38 (10% by weight) with nanogel PEG-PEI-rhodamine: Nanogel PEG-PEI-rhodamine, 20 mg (MW ˜35500; 0.56 μmol), was dissolved in 2 mL of deionized water. To it, a suspension of 2 mg (MW 392; 5.2 μmol) of SN-38 in 2 mL of methanol was added (a suspension of SN-38 in methanol was resulted after sonication). Most solids precipitated out after the mixing and the mixture was lyophilized to give a powder.

Encapsulation of SN-38 (15% by weight) with nanogel PEG-PEI-rhodamine: Nanogel PEG-PEI-rhodamine, 20 mg (MW ˜35500; 0.56 μmol), was dissolved in 2 mL of deionized water. To it, a suspension of 3 mg (MW 392; 7.8 μmol) of SN-38 in 2 mL of methanol was added (a suspension of SN-38 in methanol was resulted after sonication). Most solids precipitated out after the mixing and the mixture was lyophilized to give a powder. The above two experiments indicated that 10% and 15% of SN-38 in nanogels are not suitable for encapsulation (not all drugs are encapsulated; or the mixture is not soluble in water-methanol).

Encapsulation of tachyplesin (5% by weight) with nanogel PEG-PEI-rhodamine: Tachyplesin (H₂N—K—W—C—F—R—V—C—Y—R-G—I—C—Y—R—R—C—R—COOH (Nakamura, T. et al. J. Biol. Chem. 1988, 263, 16709-16713) was synthesized using a microwave peptide synthesizer (Discover SPS Microwave peptide synthesizer, CEM Co., Matthews, N.C.) and purified with a HPLC. A solution of 20 mg (MW ˜35500; 0.56 μmol) of nanogel PEG-PEI-rhodamine was dissolved in 2 mL of deionized water. To it, a solution of 1 mg of tachyplesin antimicrobial peptide in 2 mL of acetonitrile, was added, and the resulting nanogel solution was sonicated for 1 minute and lyophilized to give a powder, which is soluble in water.

### Example 3

With an estimated 1.15 million new cases each year, breast cancer is by far the most frequent cancer in women. It is characterized by a distinct metastatic trend to regional lymph nodes, bone marrow, lung and liver. The current cure rate of advanced or recurring breast cancer is very low. Chemotherapy is a major strategy to treat breast cancer patients along with surgery and/or radiation therapy. However, chemotherapy is limited by several drawbacks such as low bioavailability, low drug concentrations at the tumor site, systemic toxicity, lack of specificity and the development of drug resistance in tumors. Nanoparticle or nanogel delivery of therapeutic molecules represents a major improvement for more focused delivery of such therapeutic molecules.

Another avenue for increasing the specificity of delivery is via stem cells that can serve as delivery vehicles for targeting therapeutic cytokines to tumors. Stem cells isolated from the Wharton's jelly of umbilical cord, termed ‘umbilical cord matrix stem’ (UCMS) cells (Mitchell et al. 2003. Stem Cells 21:50-60) can also traffic selectively to tumors (Rachakatla et al. 2007. Cancer Gene Ther. 14:828-35). These multipotent, prenatal cells can be isolated in large numbers postnatally from an inexhaustible source. They express the ESC-like genes Oct4, Nanog and Sox2 (Carlin et al. 2006. Reprod. Biol. Endocrinol. 4:8; Weiss et al. 2006. Stem Cells 24:781-92), and a subset have the ESC surface markers SSEA3, SSEA4, and TRA1-60 (Hoynowski et al. 2007. Biochem. Biophys. Res Commun. 362:347-53). Moreover, UCMS cells elicit only minimal immune responses as shown by one-way mixed lymphocyte reactions (immunological tolerance). The preliminary data has been confirmed by a recent published report (Cho et al. 2007. Blood). It has been shown that these cells can attenuate human breast tumor growth in a mouse model when they are engineered to express a cytokine, interferon beta (Rachakatla et al. 2007. Cancer Gene Ther. 14:828-35). Here, it is proposed to merge the power of stem cells as delivery vehicles with nanotechnology by loading them with nanoparticles containing anti-cancer drugs. The stem cells will be engineered to express a suicide gene, thymidine kinase (TK). TK metabolizes the harmless pro-drug ganciclovir to form a cytotoxic chemical that will cause the stem cell to undergo apoptosis, releasing the nanoparticle-therapeutic agent payload into the tumor. The central hypothesis is that stem cells can be used as a platform for targeted delivery of therapeutic nanoparticles for breast cancer treatment, and that the therapeutic nanoparticles will achieve sustained release of high concentrations of anti-cancer therapeutics in tumors, thus regressing breast cancer. The targeted therapy is significantly effective in both primary and metastasized breast cancer. In addition this therapy is anticipated to cause considerably fewer side effects than traditional therapeutic approaches.

There is now compelling evidence that some stem cells will traffic to tumors, since signals that mediate recruitment, engraftment and proliferation of stromal cells in tumors also mediate the engraftment and proliferation of stem cells (Aboody et al. 2000. Proc. Natl. Acad. Sci. U.S. A 97:12846-51, Ehtesham et al. 2004. 6:287-93, Nakamizo et al. 2005. Cancer Res. 65:3307-18, Nakamura et al. 2004. Gene Ther. 11:1155-64). Therefore, it not surprising that there are now a number of reports showing that genetically engineered stem cells are an efficient delivery system of therapeutic proteins to cancer and other sites of inflammation (Aboody et al. 2006. Neuroncol. 8:119-26, Brown et al. 2003. Hum. Gene Ther. 14:1777-85, Ehtesham et al. 2004. Cancer Control 11:192-207, Ehtesham et al. 2002. Cancer Res. 62:7170-4, Ehtesham et al. 2002. Cancer Res. 62:5657-63, Ehtesham et al. 2002. Cancer Gene Ther. 9:925-34, Studeny et al. 2002. Cancer Res. 62:3603-8, Studeny et al. 2004. J. Natl. Cancer Inst. 96:1593-603). This could be especially relevant in cases where some therapeutic proteins given systemically cause serious adverse effects (Nakamizo et al. 2005. Cancer Res. 65:3307-18, Studeny 2002, supra, Studeny 2004, supra, Yu et al. 2003. J. Neurooncol. 64:55-61). For example, human stem cells were engineered to express IFN-β and administered to SCID mice that had malignant MDA 231 pulmonary metastastatic lesions in lunges tumors; the MSCs ‘homed’ to the tumors and suppressed growth of metastatic lesions(Rachakatla, supra). Neural stem cells transplanted into intracranial gliomas engrafted in the tumors and appeared to ‘track down’ tumor cells migrating away (Aboody 2000, supra). Neural progenitor cells isolated from bone marrow, engineered to express interleukin 4 and transplanted into mice with glioblastomas led to survival of most tumor bearing animals (Benedetti et al. 2000. Nat. Med. 6:447-50). Neural progenitor cells isolated from bone marrow (Kabos et al. 2002. Exp. Neurol. 178:288-93), and engineered with interleukin 12 (Ehtesham et al. 2002. Cancer Res. 62:5657-63), or tumor necrosis factor-related apoptosis-inducing ligand (Ehtesham et al. 2002. Cancer Res. 62:7170-4) yielded similar promising results. UCMS cells, like other the stem cells mentioned above, appear to traffic toward areas of tumor growth, and when they are engineered to secrete a cytokine, can attenuate metastatic breast cancer in a mouse model (Rachakatla, supra). Several chemokines are known to be secreted by tumors that may mediate the tropism of stem cells for them, including vascular endothelial growth factor (VEGF), transforming growth factor (TGF) family members, fibroblast growth factor (FGF) family members, platelet derived growth factor (PDGF) family members, epidermal growth factor (EGF) and IL8(Nakamura et al. 2004. Gene Ther. 11:1155-64).

Nanotechnology is a rapidly emerging drug-delivery system that makes possible the controlled release of small molecules (Duncan R. 2003. Nat. Rev. Drug Discov. 2:347-60, Vinogradov et al. 2006. Pharm. Res. 23:920-30). Nanoparticles are colloidal systems of sub-micrometer size that can be made from many different materials in a variety of compositions (van Vlerken and Amiji 2006. Expert. Opin. Drug Deliv. 3:205-16). Examples of biocompatible and biodegradable nanoparticles include poly(lactic-co-glycolic acid) (PLGA) (Berkland et al. 2004. Biomaterials 25:5649-58), poly(ε-caprolactone), and poly(β-amino esters) (van Vlerken and Amiji 2006. Expert. Opin. Drug Deliv. 3:205-16). Other nanosized systems include liposomes, polymer micelles, and nanogel polymers. Examples of the latter include Pluronic-cl-polyethylenimine (PEI) (Vinogradov et al. 2006. Pharm. Res. 23:920-30) or poly(ethylene glycol)(PEG)-PEI. (Sung et al. 2003. Biol. Pharm. Bull. 26:492-500, Vinogradov et al. 2004. Bioconjug. Chem. 15:50-60). Advantages of the nanogel system include a simpler formulation and the ability to lyophilize and store at room temperature. A downside of conventional chemotherapy includes the therapeutic drugs causing damage to healthy tumor-surrounding tissue and the drug treatment not being localized to just the tumor tissue. Incorporating nanotechnology into cancer therapy improves the ability to target the tumor because the tumor blood vessels are more permeable than other microvasculature (Duncan R. 2003. Nat. Rev. Drug Discov. 2:347-60, Matsumura and Maeda, 1986. Cancer Res 46:6387-92), resulting in an enhanced permeability and retention (EPR) effect (van Vlerken and Amiji 2006. Expert. Opin. Drug Deliv. 3:205-16). Other improvements have been gained by coating the nanoparticles with antibodies or ligands to surface molecules expressed at high levels on cancer cells (Salata 2004. J. Nanobiotechnology. 2:3). However, further improvement of specificity is needed. A recent publication suggests that a cell-mediated approach could provide a means to this end (Dou et al. 2004 Blood 108:2827-35). Although the concept is significantly different than what is proposed here, this report provides evidence that small crystals of an antiviral drug Indinavir can be internalized by macrophages and delivered in high levels to the spleen in a mouse HIV model. Thus, it might be possible to extend and enhance the power of nanotechnology by first loading the nanoparticles into stem cells, thus providing greater precision of targeted delivery. Preliminary results indicate that when a therapeutic drug is incorporated into nanoparticles, it kills cancer cells much more efficiently than if it is given alone (see Examples above). Much of this effect is probably because the nanoparticle-therapeutic drug is internalized by the cancer cells so that intracellular release has a more potent effect.

UCMS cells have been successfully prepared and characterized: Umbilical cord matrix (Wharton's jelly), the gelatinous connective tissue in the umbilical cord, is a novel source of primitive stem cells (Mitchell et al. 2003. Stem Cells 21:50-60). The cells found within the matrix of Wharton's jelly are different from those derived from umbilical cord blood. Human, porcine, canine and rat UCMS cells have been successfully isolated. Experiments revealed that these UCMS cells express stem cell markers and can be grown in vitro for long periods of time (>50 population doublings), although current focus is on cells that have been maintained for less than 20 population doublings to minimize possible genomic alterations. Several clonal populations have been isolated (data not shown) along with a line of immortalized rat UCMS cells. A subset of human UCMS cells responds to the differentiation signals in vitro and exhibits neuronal characteristics (Mitchell 2003, supra). In previous experiments, transplantation of IFN-β over-expressing human UCMS cells resulted in a significant reduction of lung metastasized tumor growth in an immunodeficient mouse model (Rachakatla, supra). These accomplishments and data indicate that IFN-β over-expressing UCMS cells should be a useful therapeutic tool in treating breast adenocarcinoma

Human UCMS cells exhibited targeted migration to lung cancer tissue: Tumor tissue consists of tumor cells, multiple stromal cells and matrix (Hall et al. 2007. Handb. Exp. Pharmacol.263-83). The tumor-supporting stroma is apparently recruited by tumor cells. It is possible that signals that mediate recruitment, engraftment and proliferation of stromal cells in tumors might also mediate engraftment and proliferation of mesenchymal stem cells such as UCMS cells. Human UCMS cells were administered to SCID mice previously injected with MDA 231 breast carcinoma cells (generous gift from Dr. I. Fidler, MD Anderson Cancer Center, Houston, Tex.) that formed metastatic lesions in the lung. The human UCMS cells, preloaded with the fluorescent dye SP-DiI, preferentially ‘homed’ to the metastatic tumor lesions (data not shown). Previous studies also indicate that bone marrow-derived MSC also specifically home to cancerous tissues (Studeny 2004, supra).

IFN-β-expressing human UCMS cells substantially attenuated growth of malignant cancer cells in vitro and lung metastasized breast cancer cell tumor in vivo: An IFN-β adenovirus vector was obtained from Dr. F Marini (MD Anderson Cancer Institute). The vector adenovirus is fiber-modified to facilitate transduction of mesenchymal cells (Studeny 2002, supra). IFN-β over-expressing human UCMS cells were prepared. The cells secrete significant amounts of IFN-β into the media (data not shown) (Rachakatla, supra). The growth alteration effect of engineered cells on the cancer cells (MDA 231 cells) was evaluated. A significant growth attenuation effect was also observed when these cancer cells were cultured with the media conditioned with the IFN-β secreting UCMS cells. The effect of IFN-β over-expressing UCMS cells and their conditioned media on cancer cell death in vitro. Both INF-β-UCMS cells and their conditioned media significantly increased cell death was also examined. Engineered UCMS cells (5×10⁵ cells) were then administered to 5 week old female C.B-17 SCID mice by weekly IV injection for three weeks, starting at 8 days after tumor inoculation. Preliminary inoculation with malignant tumor cells (IV injection, 2×10⁶ MDA 231) formed metastatic lesions in the lung. Genetically engineered human UCMS cells significantly suppressed growth of metastatic tumor burden.

Synthesis and anti-tumor activities of AQ and TT compounds: After screening the antitumor activities of a number of synthetic intermediates, it was found that substituted 1,4-anthracenediones (code name AQs) and triptycene bisquinones (code name TTs) possess potent antitumor activities (Hua et al. 2004. Tetrandedron 60:10155-63, Hua et al. 2002. Tetrahedron 60:10155-63, Hua et al. 2006. Anticancer Agents Med. Chem. 6:303-18, Wang et al. 2002. Cancer Lett. 188:73-83). The synthesis of AQ10 proceeds from a double Friedel-Crafts reaction of dihydroquinone and 4-methylphthalic anhydride. The resulting Friedel-Crafts product, AQ19, was reduced to AQ8, and was halogenated with cuprous bromide and t-butyl hydroperoxide to give AQ9. Displacement reaction of AQ9 with silver trifluoroacetate in dioxane afforded AQ10. Other active quinones such as AQ1 and AQ4 were similarly prepared (Perchellet et al. 2004. Biochem. Pharmacol. 67:523-37).

The anti-tumor activities of AQ1, AQ4, and AQ8-AQ11 are summarized in Table 1. An analog of AQ10, AQ9 (NSC 727286), has been evaluated by NCI's 60 human tumor cell lines using the SRB protein assay to estimate cell growth and viability after 2 days. The GI₅₀ (growth inhibition at 50%) values of AQ9 against HL-60, MOLT-4, SR, K562, SN12 C renal, HCT-116 colon, and MDA-MB-231 breast tumor cell lines are <10, <10, 37.1, 339, 379, 606, and 735 nM, respectively. AQ10 is less cytotoxic than AQ9 in L1210 and HL60 cell lines (Table 1).

TABLE 1
Concentrations of AQs required to inhibit by 50% (IC₅₀) the viability of L1210, HL-60 and
LL/2 tumor cells, using the MTS:PMS assay at day 4 in vitro (means ± SD, n = 3).

| | L1210 cells, IC₅₀ | HL-60 cells, IC₅₀ | LL/2 cells, IC₅₀ |
|---|---|---|---|
| Compounds | values (nM) | values (nM) | values (nM) |
| AQ1 | 42 ± 2 | 140 ± 7 | 667 ± 50 |
| AQ4 | 84 ± 6 | 243 ± 16 | 3,555 ± 330 |
| AQ8 | 29 ± 1 | 87 ± 4 | 760 ± 52 |
| AQ9 | 26 ± 1 | 79 ± 3 | 680 ± 87 |
| AQ10 | 37 ± 2 | 125 ± 7 | 494 ± 59 |
| AQ11 | 462 ± 43 | 1,260 ± 104 | Not tested |

Based on the above anti-tumor results, AQ10 was used in the stem cell-nanogel system to study anticancer effects. AQ10 has shown to initially trigger early and late markers of apoptosis and later cause internucleosomal DNA fragmentation. AQ10 is insoluble in water; however, the encapsulated nanogel-AQ10 is soluble in water and more potent than AQ10 alone.

Similarly TT compounds such as TT24 were synthesized from TT2 and evaluated for their antitumor activities (Hua et al. 2004. Tetrandedron 60:10155-63, Hua et al. 2002. Tetrahedron 60:10155-63, Hua et al. 2006. Anticancer Agents Med. Chem. 6:303-18, Wang et al. 2002. Cancer Lett. 188:73-83). Selective bromination of TT2 with N-bromosuccinimide (NBS) in DMF followed by addition of allylmethylamine and removal of the allyl protecting group with Pd(PPh₃)₄ afforded TT24. TT24 (NSC 727284-K), has been evaluated by NCI's 60 human tumor cell lines using the SRB protein assay to estimate cell growth and viability after 2 days. The GI₅₀ (growth inhibition at 50%) values of TT24 against MDA-MB-231, T47D, and NCI/ADR-RES breast tumor cell lines are 1.41, 1.44, 1.55 μM, respectively. TT24 inhibits L1210 cells with IC₅₀ value of 48 nM.

Synthesis of Non-toxic nanogel PEG-PEI: Non-toxic PEG-PEI nanogel was synthesized as described above in Example 1.

Features of the Stem Cell/Nanogel/Therapeutic system. The UCMS cells have properties that suggest that they should be well-tolerated as allogeneic grafts. One-way mixed lymphocyte reactions (MLR) of human umbilical cord matrix (hUCMS) cells taken from two different passage numbers were performed. The proliferation of T cells was determined in the absence of stimulator cells, in the presence of autologous irradiated peripheral blood mononuclear cells (PBMCs) (isotypic stimulation represents a negative control), and in the presence of allogeneic irradiated PBMCs (allogeneic stimulation represents a positive control), and in the presence of hUCMS cells (P5 or P9). The stimulator cells were tested at densities of 5,000, 10,000, or 20,000 per well.

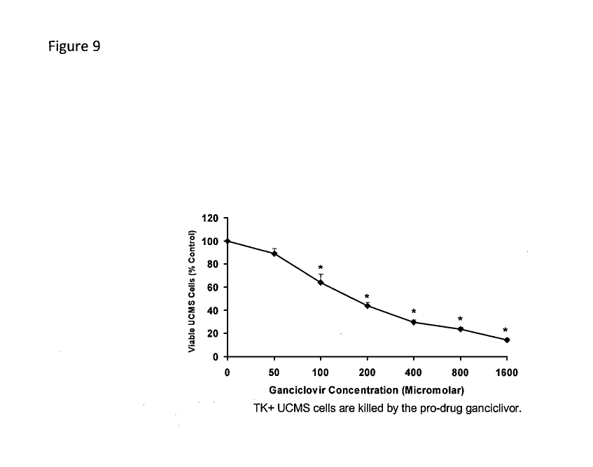

UCMS cells have been successfully engineered for stable expression of HSV-thymidine kinase. FIG. 9 shows decreased cell number following exposure of TK+UCMS cells to the pro-drug, Ganciclovir at a dose range of 0 μM to 1600 μM concentration. Thus, the suicide gene system is effective; after GCV administration to mice bearing tumors into which the UCMS-NG-TH (UCMS cells containing nanogels loaded with therapeutic agents) have trafficked, the NG-TH will be released into the tumor interstitium when the stem cells undergo apoptosis.

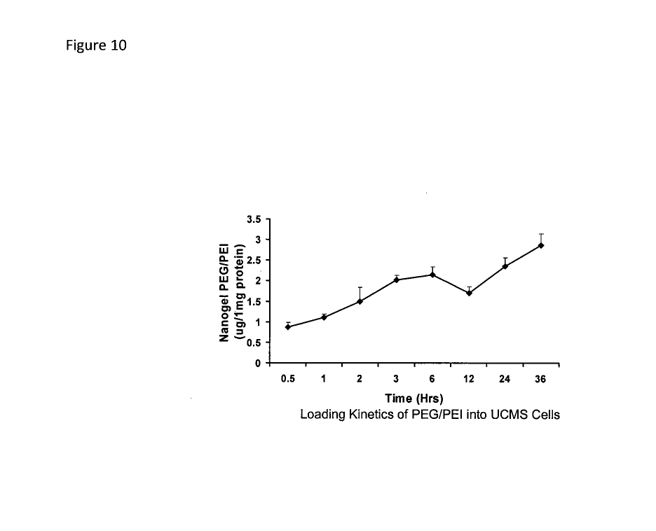

FIG. 10 shows nanoparticle loading kinetics over a period ranging from 30 minutes to 36 hours. These data show that the threshold loading of nanoparticles into UCMS. 4.7% and 4.6% of the total nanoparticles added to the stem cells was attained at 24 and 36 hour time points, respectively.

The results from the MTT assay showed that nanogel PEG-PEI (˜6.8:1, methylene protons ratio) alone is not toxic to cancer cells. AQ10 dissolved in DMSO at 4.2 μM showed a significant effect on cancer cells. In contrast, cancer cells incubated with 1% AQ10-nanogel PEG-PEI significantly decreased viable cell numbers at 0.06, and 0.1 mg dose per ml of medium, compared to nanogel PEG-PEI and AQ10 in DMSO. These results indicate that AQ10 when incorporated into nanogel PEG-PEI is more toxic to cancer cells than AQ10 alone.

Determination of the optimal nanoparticle/therapeutic combination to effect the greatest growth inhibition and viability reduction of MDA-231 and other cancer cell lines in vitro. Thymidine kinase (TK) converts ganciclivor (GCV) to GCV monophosphate, which is further phosphorylated by cellular kinases to toxic GCV-triphosphate. Mammalian cells lack TK; thus, ganciclovir causes toxic effects only in cells transfected with TK (Lumniczky and Safrany, 2006. Pathol. Oncol. Res. 12:118-24). The purpose of arming the stem cells with the suicide gene is trifold: 1. effect release of the therapeutic nanoparticles from the stem cells in a controlled manner after they have trafficked to the tumors, 2. kill tumor cells via a bystander effect, and 3. to remove the stem cells themselves to ensure that they might not have long term deleterious effects. Several small molecule anti-cancer compounds including TT24, AQ10, Doxorubicin (Sigma) and Cisplatin (Sigma) incorporated into nanogel (henceforth referred to as nanogel-therapeutic agent (NG-TH) will be tested. Cisplatin and doxorubicin are widely used chemotherapeutic agents that have many severe systemic side effects. TT24 is a tryptycene bisquinone that has potent in vitro mitochondrial-mediated anti-tumor and apoptosis inducing properties. The anthraquinone derivative AQ10 also causes apoptosis. The hypotheses to be tested are: 1. Nanogel/AQ delivered via UCMS cells when co-cultured with mammary cancer cells in vitro will cause the greatest inhibition of cancer cell growth of all the NG-TH (nanogel+various therapeutic agents) tested, and 2. Increased apoptosis is a major mechanism for this effect.

UCMS cells will be isolated as previously described (Mitchell 2003, supra); they will be propagated in ‘Defined Media’ (see General Methods). UCMA cells have been engineered to stably express TK using a commercially available plasmid containing TK gene (Addgene) and the Nucleofector system (Amaxa). PEG-PEI nanogel/rhodamine+/−therapeutic agents are prepared as described above. Stem cells will be loaded by co-incubation with 0.025 mg/ml nanoparticles in media for 6 hours with PEG-PEI nanogel/therapeutic agent (NG-TH) prepared as described above in the Hua lab. A co-culture system in soft agar (colony assay) will be utilized that features a three-dimensional tumor-like colony growth (see General Methods). Briefly, UCMS-NG-AQ10 (or control, unloaded cells) and MDA 231 cells (2−5×10⁴ cells/well of each cell type) will be suspended in 1 ml of the defined medium containing 0.4% agar and placed on top of 0.8% agar layer. The cells will be incubated at 37° C. with 5% CO₂ for 8-10 days for growth of colonies. GCV will be added when colonies become visible. Colonies greater than 600 μm² will be counted by an automated colony counter (Olympus CKX41 equipped with computer automated motor-drive stage and analysis system, St Louis, Mo.). This colony assay consisting of co-cultured breast cancer-stem cell-NG-TH is especially advantageous because it is a three-dimensional colony with both cell types that approximates the situation in vivo in the tumors. For Western blot analysis of caspases and other proteins cancer cells (bottom chamber) will be co-cultured in the transwell culture plate with UCMS-NG-AQ10 (upper chamber)(see General Methods). In addition, standard co-culture cancer cell-stem cell will be used to allow flow cytometry analysis. The cancer cells are loaded with the green-fluorescent dye CFDA (Molecular Probes/Invitrogen). Cells will be seeded at 10,000 UCMS cells+/−nanogel+/−NG-TH and 50,000 MDA-231 cancer cells. Although the work using human breast cancer cells has involved MDA-231 to date, it is also possible to analyze other breast cancer cell lines such as MCF-7 and T47D. After 24 hours of co-culture, GCV is added to the media in the GCV groups. Co-cultures will be observed and photographed daily. Seventy two hours after addition of pro-drug, viable cancer cell numbers will be analyzed using the MTT assay (Roche). All experiments are done in triplicate and repeated at least three times. Flow sorting will be used as previously described (Weiss et al. 2006. Stem Cells 24:781-92) to separate CFDA-loaded cancer cells from the unlabeled stem cells. Some of the sorted cancer cells will be treated with Vindilov's solution (50 m/ml propidium iodide in PBS/TritonX for 20 minutes and subjected to flow cytometry to analyze % dead cells. Some of the sorted cancer cells will then be further analyzed for early apoptosis using the Annexin V method (see General Methods), or for late apoptosis using Western blotting for activated caspases as described in the General Methods section.

Nanogel/AQ delivered via UCMS cells when co-cultured with mammary cancer cells in the presence of GCV will cause the greatest inhibition of cancer cell growth of all the NG-TH formulations tested. This result will be indicated if stem cell/NG/AQ treatment results in a lower number of viable cells than the other treatment groups. Increased apoptosis is a major mechanism for this effect. This result is indicated if cancer cells cocultured with UCMS/NG/AQ show increased activated caspases within the cancer cells in Western blot analysis than cancer cells incubated with stem cells alone with or without GCV, stem cells with empty NG with or without GCV, or cancer cells with no treatment.

Determination of whether nanotechnology can be merged with stem cell targeted delivery to mediate targeted attenuation of breast tumor growth in mouse lung. Many small molecule anticancer compounds shown to have enormous potential in the laboratory are not being used due to unacceptable deleterious side effects. Stem cell homing is merged with nanotherapy to enhance both delivery systems. Moreover, many small potent small molecules such as the AQ and TT families are quite insoluble; encapsulating them in nanogel is a means to avert this problem. The combination of nanogel-therapeutic drug (NG-TH) determined as described above to have the greatest ability to induce MDA-231 cell death in vitro (henceforth referred to as ‘nanogel-optimal therapeutic or NG-OTH) will be utilized in this specific aim. A mouse model identical to the model described above to show a therapeutic effect after targeted delivery of beta interferon will be used (Rachakatla, supra). It has previously been shown that the UCMS cells engrafted selectively near or within MDA-231 metastatic human breast carcinoma tumors in the lungs of SCID mice after they were transplanted systemically, and they exerted a significant therapeutic effect when they were engineered to synthesize a cytokine (Rachakatla, supra). As described above, the UCMS cells have been engineered to express the suicide gene TK, so GCV will be administered to cause the stem cells to undergo apoptosis and release the nanoparticles into the tumor interstitium. Since it has been shown above that the anticancer drug AQ10 has a more potent effect when it is added to cancer cells in PEG-PEI nanogel than when added alone, it is expected that small molecules will be more potent when delivered in vivo in nanoparticles (presumably because they are endocytosed by the cancer cells). MDA231 lung carcinoma cells will be transplanted into SCID mice followed by transplantation of human UCMS cells. The hypotheses to be tested are as follows: 1.I.V. TK+UCMS cells delivering nanoparticle-optimal therapeutic (NP-OTH; determined from specific aim 1) will reduce tumor burden more than any other treatment. This result will be indicated if IV stem cells with NP-OTH and GCV treatment reduce tumor area significantly more than all the other groups analyzed (ANOVA followed by the Newman-Keuls Post Hoc procedure). 2. The major mechanism of tumor attenuation is via apoptosis. This result will be indicated if Western blot analysis indicates significantly greater activated caspases in tumors treated with UCMS-NG-AQ10 than is seen for other groups (ANOVA followed by post-hoc testing). In situ apoptosis results showing increased numbers of positive cells in tissue sections will further support this hypothesis.

Methods: Cell preparation and tumor inoculation: Human UCMS cells that have been engineered to express TK will be loaded with nanogel particles that contain the therapeutic agent determined to be optimally effective. These nanoparticles will be prepared as described above. Female 5 week old CB 17 SCID mice (Charles River laboratories, Maryland) will be held for 1 week after arrival to allow them to acclimate. MDA231 cancer cells and stem cells are transplanted without anesthesia into the lateral tail vein using sterile conditions. Cells are trypsinized (0.025% trypsin EDTA, Invitrogen), fresh media added to inactivate the trypsin, counted using a hemocytometer, pelleted by low-speed centrifugation and resuspended in PBS. Cells are held at 37 C until used. NG-rhodamine-OTH is added to the cells at 0.025 mg/ml media. The primary means of identification of transplanted cells is via rhodamine-labeled nanoparticles in them, but as an additional labeling method the cells can be loaded with the green-fluorescing dye CFDA-SE (Invitrogen). Cells without NG will be loaded with CDFA-SE. Animals are randomly assigned to experimental groups (N=10 per group). Groups are shown in Table 3. For tumor inoculation, 1.0×10⁶ MDA231 cells are given via the lateral tail vein. UCMS cells (with or without various therapeutic payloads)(0.5×10⁶) are transplanted on day 8 after tumor inoculation. GCV is administered four days after transplant of tumor cells. The stem cell transplant is repeated twice at 1 week intervals subsequent to the first transplant (with pro-drug administration four days after each transplant), and mice are sacrificed by CO₂ inhalation and cervical dislocation one week after the last transplant. Any mice demonstrating excessive hemorrhage, open wound infections or prostration will be removed from the experiment and euthanatized early. All in vivo experiments will be carried out under proper IACUC and IBC institutional approval has been obtained.

Tissue collection and tumor burden analysis. Lung weights of control and tumor-bearing animals will be measured to estimate tumor burden. Lungs are snap-frozen in iso-pentane in liquid nitrogen for histological analysis and/or immunohistochemistry. Other organs including spleen, liver, kidney, and bone marrow are also harvested. After examination for gross lesions; they will be analyzed histologically for lesions as well. Tissues are sectioned on a cryostat at 10-12 μm. To clearly delineate MDA 231 tumors in mouse lung, tissue sections are washed with phosphate buffered saline-0.2% Triton X-100 (PBS TX) and fixed with 70% ethanol and acetone (1:1). This is followed by washing with three changes of PBS TX.

Analysis of area of lung occupied by tumors: To more clearly delineate tumor cells, tissue sections are blocked with 5% normal goat serum in PBS TX for 30 minutes, followed by incubation with anti-human mitochondrial antibody (1:1000, Chemicon, Calif.), in PBS TX overnight. The tissues are then washed three times with PBS TX and incubated with Alexa Fluor 488 conjugated secondary antibody (1:1000, Molecular Probes, Calif.) for 3 hours. The tissues are incubated for 30 min in Hoechst 33342 (10 mg/ml, Sigma, Mo.) nuclear counter stain, followed by a triple rinse with PBS TX. The antigens are localized using epifluorescence microscopy (Nikon Eclipse, Boyce Scientific Inc. MO) and images were captured using a Roper Cool Snap ES camera and Metamorph 7. A departmental confocal microscope will be used to verify findings. Random tissue sections from each group of SCID mice are taken to measure the tumor area in the lungs. Immunohistochemistry for anti-human mitochondria outlines the MDA 231 tumors with green fluorescence. The area (in square micrometers) occupied by the tumor is calculated from 10-15 200× fields for each group of mice using the MetaMorph 7 image analysis system.

In vivo apoptosis evaluation: An apoptosis detection kit will be used to label cells undergoing programmed cell death following the manufacturer's protocol (APO-BRDU-IHC; Chemicon). The number of tumor cells undergoing apoptosis within ten high magnification (400×) fields will be counted and the mean number determined for each animal carrying tumors. In addition, proteins extracted from tumors will be subjected to Western blot analysis for activated caspases (see General Methods).

It is expected that I.V. TK+UCMS cells delivering nanoparticle-optimal therapeutic will reduce tumor burden more than any other treatment. This result will be indicated if IV stem cells with NP-OTH and GCV treatment reduce tumor area significantly more than all the other groups analyzed (ANOVA followed by the Newman-Keuls Post Hoc procedure). The major mechanism of tumor attenuation is via apoptosis. This result will be indicated if Western blot analysis indicates significantly greater activated caspases in tumors treated with UCMS-NG-AQ10 than is seen for other groups (ANOVA followed by post-hoc testing).

General Methods. Tissue culture of human umbilical cord matrix stem cells and MDA 231 cells: Human umbilical cord matrix stem (UCMS) cells are harvested from term deliveries at the time of birth with the mother's consent. The methods to isolate and culture human UCMS cells were previously described (Mitchell et al., supra). UCMS cells are maintained in ‘Defined Medium’ (DM) (a mixture of 56% low glucose DMEM (Invitrogen), 37% MCBD 201 (Sigma; St. Louis, Mo.) and 2% fetal bovine serum (FBS, Atlanta Biologicals Inc, Georgia) containing 1× insulin-transferrin-selenium-X (ITS-X, Invitrogen, CA), 1× ALBUMax 1 (Invitrogen, CA), 1× Pen/Strep (Invitrogen, CA), 10 nM dexamethasone (Sigma, Mo.), 100 μM ascorbic acid 2-phosphate (Sigma, Mo.), 10 ng/ml epidermal growth factor (EGF, R&D systems, Minneapolis), and 10 ng/ml platelet derived growth factor-BB (PDGF-BB, R&D systems, MN) at 37° C. in a humidified atmosphere containing 5% carbon dioxide. MDA 231 human breast carcinoma cells that metastasize to the lung in nude mice were obtained from M.D. Anderson Cancer Center (Houston, Tex.) as a gift from F. Marini. They are maintained in the same media as that described for UCMS cells above.

Immunohistochemical staining: For immunofluorescence, tissue sections are washed with phosphate buffered saline (PBS) and fixed. This is followed by washing with three changes of PBS. Tissue sections are blocked with 10% normal blocking serum (goat serum) in PBS for 30 minutes, and followed by incubation with primary antibody, (anti-human mitochondrial antibody) (1:1000, Chemicon), in PBS for overnight. The tissues are then washed three times with PBS and incubated with Alexa Fluor 488 conjugated secondary antibody (Molecular Probes, Calif.) for 3 hours. The tissues are incubated for 30 min in Hoechst 33342 (5 μl/ml of a 1 mg/ml solution, Sigma, Calif.) as a counter-stain to label the nuclei followed by a triple rinse with PBS. The antigens are localized using epifluorescence microscopy (Nikon Eclipse) and images are captured using a Roper Cool Snap ES camera and Metamorph 7. Confocal microscopy will be used to verify the findings.

MTT assay: The MTT assay will be used to determine numbers of viable cells. The MTT assay labels metabolically active cells and will be performed using the manufacturer's protocol. The absorbance of the samples is detected using a microtiter plate reader with fomazan product at 570 nm, and the reference wavelength at 750 nm.

Identification of human umbilical cord matrix stem cells: For transplanted UCMS cell identification, the fluorescent dye SP-DiI (Molecular Probes) is dissolved in dimethylsulphoxide (DMSO) at a concentration of 5 mg/ml. SP-DiI dye is added to culture medium to a final concentration of 10 μg/ml and human UCMS cells are labeled by adding 10 ml of medium with SP-DiI in a T-75 flask for 24 hours. Then, cells are washed with PBS, incubated with dye-free medium for 4 hours, and used for experiments. Alternatively, cells will be loaded with CFDA-SE (caboxyfluorscein diacetate-succinimidyl ester; Molecular Probes-Invitrogen), which is excited using the fluoroscein filter and results in green fluorescence. Briefly, cells are incubated with pre-warmed PBS containing 10 μM CFDA for 15 minutes at 37 C. Then, the CFDA is replaced with fresh pre-warmed media and incubated for at least 30 minutes at 37 C prior to imaging or transplantation.

In vitro apoptosis analysis: The co-cultured stem cells will have been loaded with CFDA and sorted from the co-culture system using flow-sorting as follows. Briefly, cells will be dissociated in trypsin/EDTA. Fluorescence-activated sorting will be done using ultra violet laser and fluorescence will be measured using fluorescence filter. Flow sorted cancer cells from co-cultures will be subjected to analysis for early apopotosis (Annexin V assay) or late apoptosis using Western blotting for activated caspases. For the analysis of early apoptosis, cancer cells will be prepared for Annexin V-FITC FACS analysis, according to manufacturer's protocol (Annexin V-FITC Apoptosis Detection Kit; Bio Vision Inc. Mountain View, Calif.). The cells are first washed with PBS, trypsinized and resuspended in DM containing 5% FBS. Approximately 2×10⁵ MDA231 cells from various treatments are subjected to evaluation of apoptosis index by the Annexin V-FITC Apoptosis Detection Kit (Bio Vision Inc. Mountain View, Calif.). The FACS analysis is carried out by FACSVantage SE flow cytometer (BD Biosciences, San Jose, Calif.).

Western blot analysis: Western blot analyses will be performed on protein isolated from cancer cells. Briefly, equal amounts of protein samples will be run on SDS-PAGE gels and electroblotted onto nitrocellulose membrane and the membranes will then be blocked with 5% milk plus 1% bovine serum albumin to reduce nonspecific binding for overnight at 4° C. The membrane will be incubated for 1 hour with primary polyclonal antibody against Caspase 8 and Caspase 3 proteins. After a brief wash the membrane will be incubated with secondary antibody (goat-anti-rabbit IgG) conjugated to horseradish peroxidase (Promega) used at 1:5000 in TBS-NP40 buffer. Detection of immunopositive bands will be performed with the Amersham ECL kit (Biocompare) according to the manufacturer's instructions. Actin will be used as loading control for densitometric quantification and proper positive and negative controls will be used in each run to show the specificity of antigen-antibody interactions.

Tumor colony assay: UCMS cells (loaded with NG or unloaded) will be grown in a six well plate culture dish. Once UCMS cells were grown to approximately 30% confluent, 1 ml 0.8% agar in defined medium for UCMS cells will be poured into the dish (bottom layer). MDA 231 cells (2−5×10⁴ cells/well) will be suspended in 1 ml of the defined medium containing 0.4% agar and plated on top of the bottom agar layer. The cells are incubated at 37° C. with 5% CO₂ for 8-10 days for growth of colonies. Colonies greater than 700 μm² will be counted by an automated colony counter (Olympus CKX41 equipped with computer automated motor-drive stage and analysis system, St Louis, Mo.). The two cell types will also be mixed and added together to the 0.8% agar in Defined medium so that three dimensional colonies form that contain both UCMS-NG-OTH or UCMS-NG, so that they are in close proximity to MDA231 cells when GCV is added when colonies become visible. The colony assay is a significantly good procedure to evaluate the effect of nanoparticle-loaded stem cells on malignant cancer cell growth since this method quantitatively evaluates tumor growth.

### Example 4

This example provides a protocol for loading of acetylated PEG/PEI particles along with Lipofectamine 2000.
1. Plate cells at 30-50% confluence.
2. 24 hours later, weigh acetylated PEG/PEI particles and dilute at a concentration of 0.1 mg/ml of DMEM.
3. Add 5 ul/ml of Lipofectamine 2000 to the PEG diluted solution and leave for 20 minutes.
4. Replace the original medium from the plated cells and add PEG and Lipofectamine diluted solution to the cells
5. Incubate the cells overnight.
6. Change the DMEM medium with cells regular medium.
7. Visualize the cells under fluorescent microscope.

### Example 5

This Example provides a protocol for isolation of neutrophils (PMN's) and loading with nanogel.
1. Mix equal quantities of fresh Heparinized Blood with equal volumes of 1× PBS
2. Layer the diluted blood over Ficoll-Hypaque gradient (Density 1077) in a polypropylene tube and centrifuge at 400×g for 30 min at room temperature (19-220 C)
3. Following centrifugation, three fractions are formed:
   Fraction 1. Plasma
   Fraction 2. Peripheral Blood Mononuclear Cells (Plasma-Ficoll interphace)
   Fraction 3. Granulocytes and Red Blood Cells (Pellet)
4. Using a Pasteur pipette the top 2 layers are removed; alternatively the pipette is poked through the top two layers and the pellet is recovered (gently).
5. The pellet is washed three times with 1× PBS by centrifugation at 30× g for 10 min.
6. The pellet is then suspended in C1NH4 buffer (RBC lyses buffer) for 10 min at room temperature and centrifuged at 30× g for 10 min.
7. Remove the supernatant and wash the pellet thrice with 1× PBS
8. Following washes the pellet is suspended in fresh media.

### Nanogel Loading

9. Nanogel particles were added at 0.05 and 0.1 mg/ml for two-six hours.
10. Cells are washed in sterile PBS, centrifuged and after removal of supernatant (process repeated once), analyzed and photographed on an epifluorescent microscope.

### Example 6

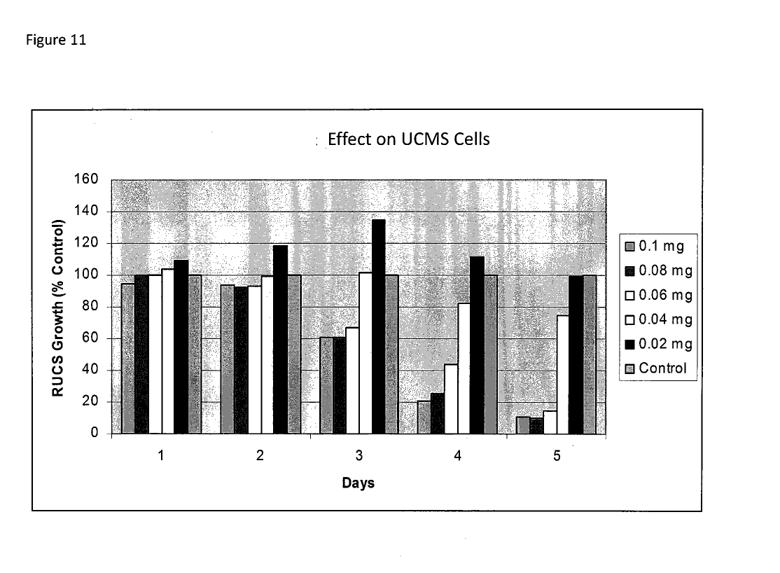

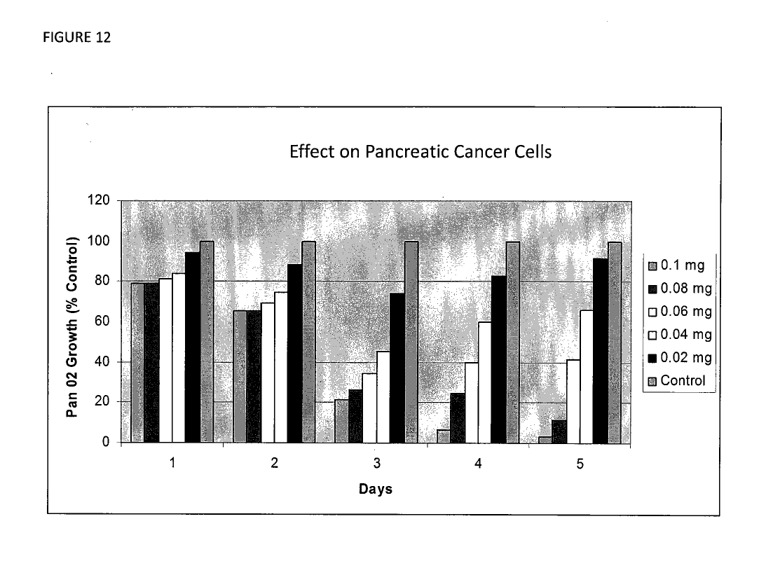

This Example documents the effect of various concentration of Triton X on cell viability. RUCS or Pan 02 cells were loaded with NG-AQ5% (control, no Triton X) or NG-AQ5%-TX1% (0.1, 0.08, 0.06, 0.4. 0.02 mg Triton X). Cells were plated at 5000/96well for RUCS and 7400/96well for Pan 02 cells. When cells reached ˜70% confluency, nanoparticles were added and incubated. Cellular toxicity was measured using a MTT assay. The results are presented in FIGS. 11 and 12. As can be seen, cells loaded with a sufficient amount of the detergent (1.0, 0.08 or 0.06 mg) could be programmed to undergo apoptosis after 72 or 96 hours.

### Example 7

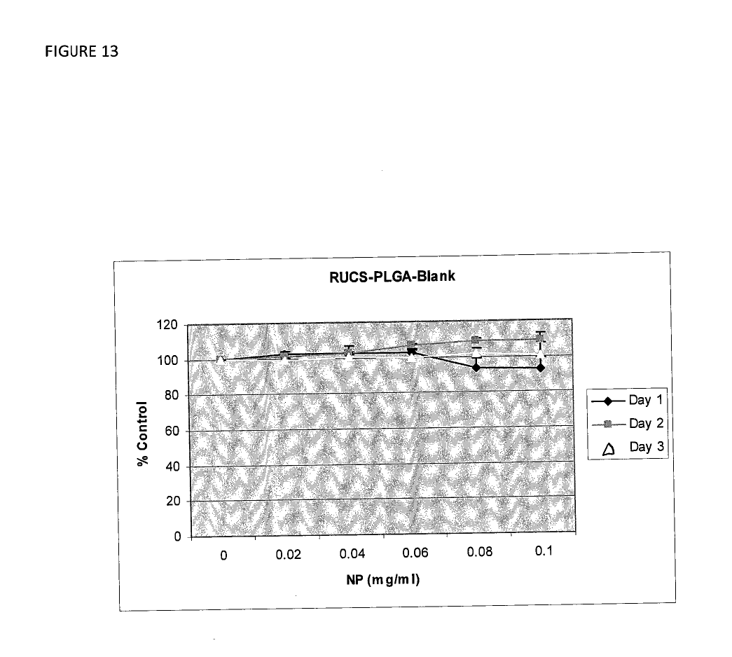

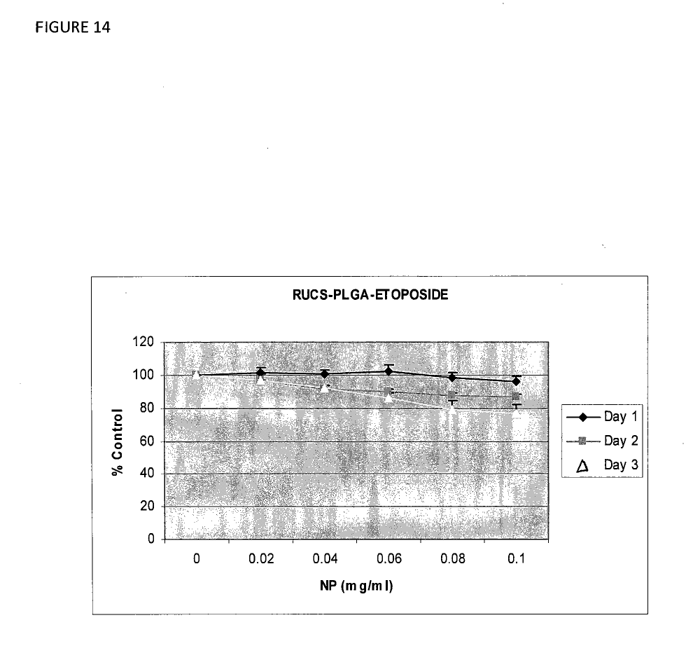

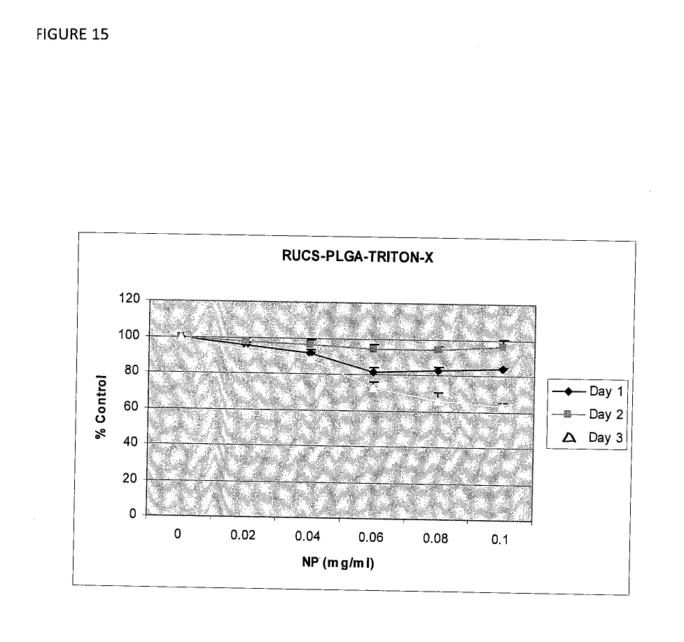

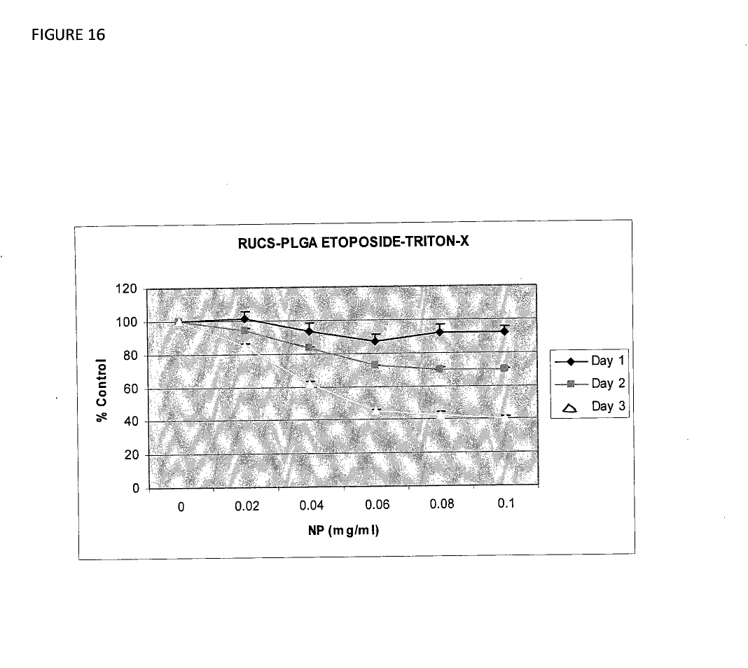

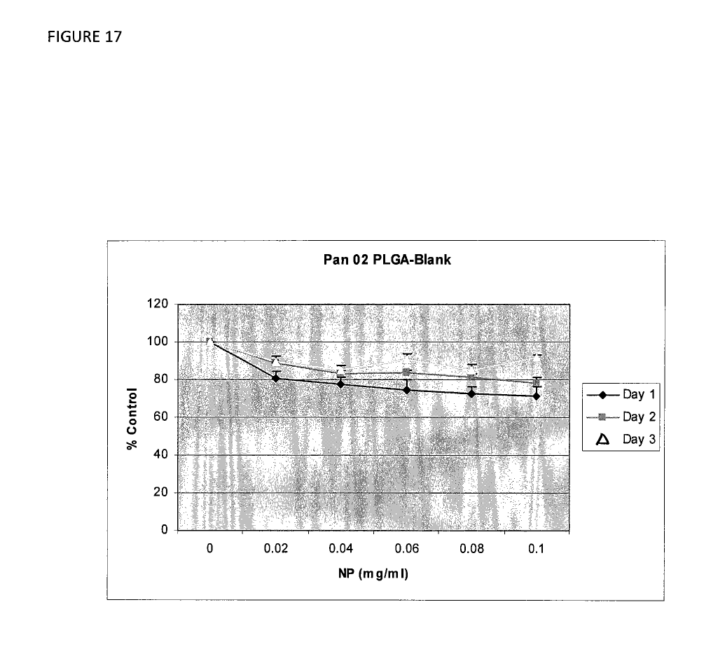

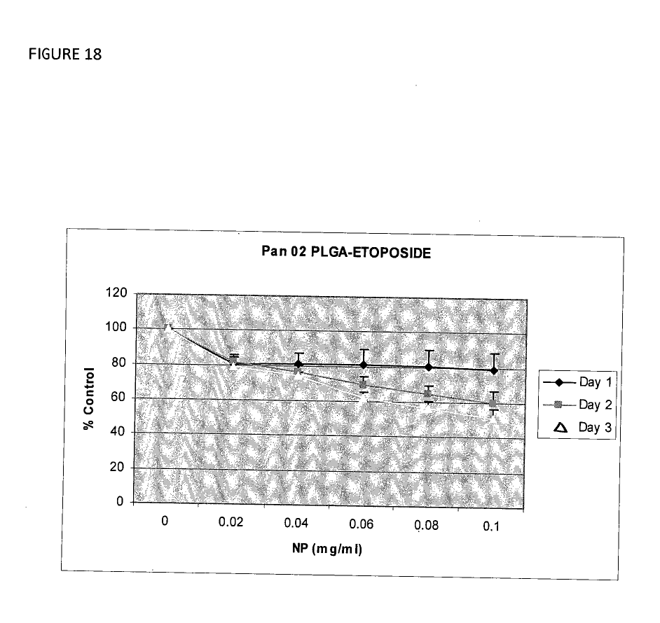

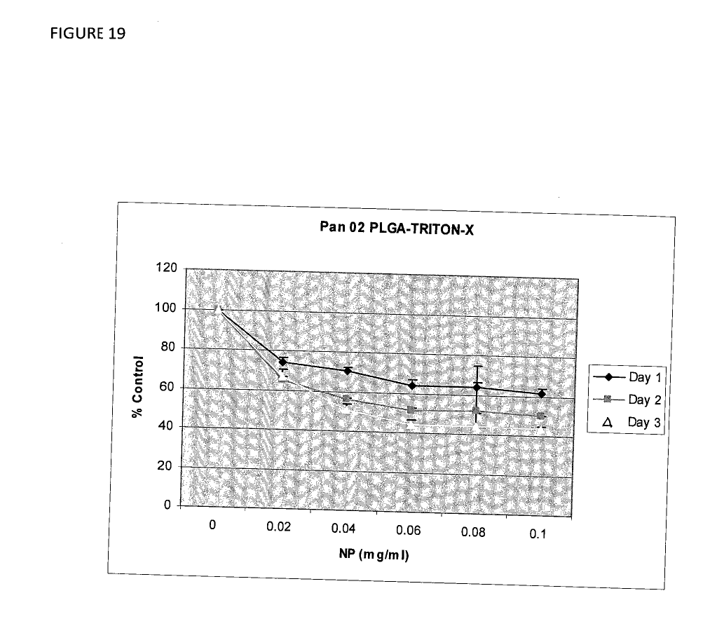

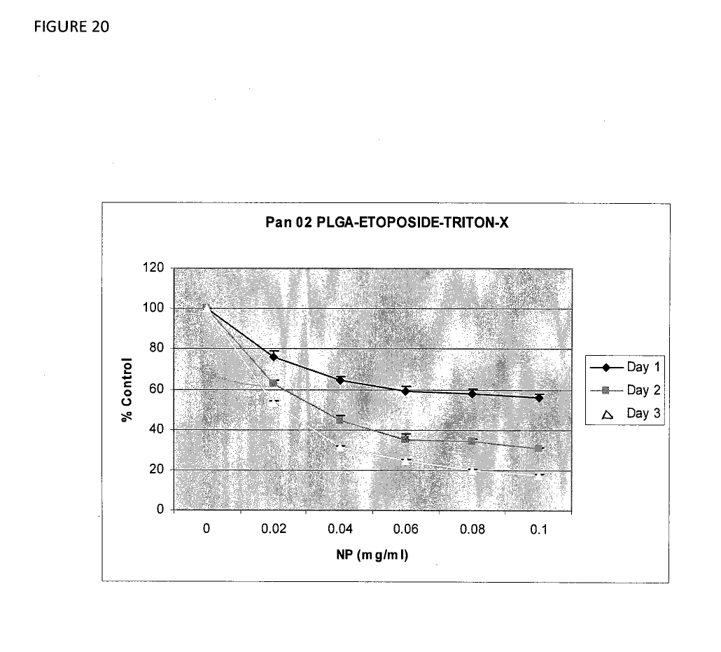

This example shows the effect of PLGA nanoparticles loaded with Etoposide+/_ triton x on RUCs (rat umbilical cord matrix stem cells) and PAN02 (pancreatic carcinoma cells), clearly showing a powerful additive effect of the two on the pancreatic cancer cells (FIGS. 13-20; cell viability measured by MTT assay and expressed as % control on X axis, mg/ml nanoparticle used to load cells expressed on Y axis). Photomicrography clearly demonstrated the loading of stem cells with PLGA-rhodamine particles and that the rat UCMS cells migrated to the interstitium of the lung only two days after administration (data not shown).

For preparation of PLGA nanogel, Poly(DL-lactic-co-glycolic acid) (50:50) (PLGA, inherent viscosity 0.89, Mw ˜150 kDa) is used. PLGA nanoparticles coated with PVAm (Polyvinylamine) and containing Doxorubicin(nano/dox) or Etoposide are prepared by the solvent diffusion method. In some cases, during incorporation of doxorubicin or other chemotherapeutics such as TT24, AQ, SN-38 etc., rhodamine or Cy5 is incorporated to assist nanoparticle tracking in vivo. Stem cells are loaded by co-incubation with 0.025 mg/ml-0.1 mg/ml nanoparticles in media for 18 hours with PLGA nanoparticle/dox.

1. A composition comprising:
an in vitro culture of cells, said cells comprising a nanogel comprising an active agent and a lytic agent, wherein said lytic agent is provided in an amount sufficient to cause lysis of said stem cells at a predetermined time.

2. The composition of claim 1, wherein said cells are stem cells.

3. The composition of claim 2, wherein said stem cells are selected from the group consisting of pluripotent stem cells and multipotent stem cells.

4. The composition of claim 2, wherein said stem cells are selected from the group consisting of embryonic stem cells and adult stem cells.

5. The composition of claim 2, wherein said stem cells are umbilical cord matrix stem cells.

6. The composition of claim 1, wherein said cells are immune system cells.

7. The composition of claim 6, wherein said immune system cells are selected from the group consisting of leukocytes and lymphocytes.

8. The composition of claim 7, wherein said leukocytes are selected from the neutrophils, macrophages, dendritic cells, mast cells, eosinophils, basophils, monocytes and natural killer cells.

9. The composition of claim 7, wherein said lymphocytes are selected from the group consisting of helper T cells, killer T cells, and B cells.

10. The composition of claim 1, wherein said lytic agent is a detergent.

11. The composition of claim 10, wherein said detergent is selected from the group consisting of Triton X-100 and Tween-20.

12. The composition of claim 1, wherein said cells comprise a suicide gene and said lytic agent is a pro-drug that is activated by the gene product of the suicide gene.

13. The composition of claim 12, wherein said suicide gene is thymidine kinase and said pro-drug is ganciclivor.

14. The composition of claim 1, wherein said nanogel comprises a polymer selected from the group consisting of PEG, PEI, PGA, PLGA and PLA and combinations thereof.

15. The composition of claim 1, wherein said nanogel is a PEG/PEI nanogel.

16. The composition of claim 15, wherein said PEG/PEI nanogel has a methylene proton ratio (CH₂O:CH₂N) of about 6.0:1 to about 8.0:1.

17. The composition of claim 1, wherein said predetermined time is from about 36 to 96 hours.

18. The composition of claim 1, wherein said active agent is selected from the group consisting of a therapeutic protein, a therapeutic compound, an antibiotic compound, and an antiviral compound.

19. The composition of claim 18, wherein said therapeutic protein is an antimicrobial polypeptide.

20. The composition of claim 18, wherein said therapeutic compound is a chemotherapeutic compound.

21. A nanogel comprising a therapeutic agent and a lytic agent, wherein said lytic agent is provided in an amount sufficient to cause cell lysis at a predetermined time following introduction into a cell.

22. A composition comprising:
an in vitro culture of stem cells, said cells comprising a nanogel comprising an active agent.

23. A process for making a targeted therapeutic cell composition comprising:
providing a culture of cells and a nanogel comprising a therapeutic agent and a lytic agent, wherein said lytic agent is provided in an amount sufficient to cause lysis of said cells at a predetermined time;
loading said nanogel into said cells to provide nanogel-loaded cells.

24. A method for treating a subject comprising:
administering to a subject in need of treatment the composition of claim 1.

25. A non-toxic nanogel composition comprising particles comprising PEI having a size of from about 0.1 to about 200 nm, wherein said particles are non-toxic when introduced into a cell.

26. The non-toxic nanogel composition of claim 25, further comprising a blocking agent present in a sufficient concentration to block amino groups on said PEI so that said PEI is non-toxic to cells.

27. The non-toxic nanogel composition of claim 26, wherein said blocking agent is PEG and said PEG is present in said composition so that said nanogel has a methylene proton ratio (CH₂O:CH₂N) of about 6.0:1 to about 8.0:1.

28. The non-toxic nanogel composition of claim 25, wherein said nanogel further comprises PEG cross-linked with said PEI and a blocking moiety.

29. The non-toxic nanogel composition of claim 28, wherein aid blocking agent is selected from the group consisting of an alkyl moiety, and alkenyl moiety, an aryl moiety, and acetyl moiety, and rhodamine.

30. The non-toxic nanogel composition of claim 29, wherein said blocking agent is attached to said nanogel via an amino group on said nanogel.

31. The non-toxic nanogel composition of claim 25, wherein said nanogel composition is lyophilized.

32. The non-toxic nanogel composition of claim 25, wherein said nanogel composition further comprises a labeling agent.

33. A composition comprising:
an in vitro culture of immune system cells, said cells comprising a nanogel comprising an active agent.
